(19) United States
(12) Patent Application Publication
(10) Pub. No.: US 2010/0063583 A1
(43) Pub. Date: Mar. 11, 2010

(54) USE OF SOLUBLE EPOXIDE HYDROLASE INHIBITORS IN THE TREATMENT OF INFLAMMATORY VASCULAR DISEASES

(75) Inventors: Yi-Xin Wang, Lafayette, CA (US); Le-Ning Zhang, Pleasant Hill, CA (US); Gabor M. Rubanyi, San Diego, CA (US)

Correspondence Address: Swiss Tanner, P.C.;P.O. Box 1749, Four Main Street, Suite 100, Los Altos, CA, 94022, US

(73) Assignee: Arete Therapeutics, Inc.

(21) Appl. No.: 12/541,778

(22) Filed: Aug. 14, 2009

Related U.S. Application Data
(60) Provisional application No. 61/093,177, filed on Aug. 29, 2008.

(51) Int. Cl. A61F 2/82; A61P 29/00; A61K 31/4468; A61K 31/5375; A61K 31/4545; A61K 31/17

(52) U.S. Cl. 623/1.38; 514/325; 514/237.5; 514/318; 514/329; 514/598; 514/588

(57) ABSTRACT

Disclosed herein are compositions and methods for treating inflammatory vascular diseases. Examples of inflammatory vascular disease include, but are not limited to, in-stent stenosis, coronary arterial diseases (CAD), angina, acute myocardial infarction, acute coronary syndrome, chronic heart failure (CHF), peripheral arterial occlusive diseases (PAOD), critical limb ischemia (CLI), cardiac, kidney, liver and intestinal ischemia, renal failure, cardiac hypertrophy, atherosclerosis, abdominal aortic aneurysm, vasculitis, carotid artery stenosis.

### CROSS-REFERENCE TO RELATED APPLICATIONS

This application claims the benefit under 35 U.S.C. §119(e) of U.S. Provisional Patent Application Ser. No. 61/093,177, filed on Aug. 29, 2008, which is incorporated herein by reference in its entirety.

### FIELD OF THE INVENTION

Disclosed herein are compositions and methods using sEH inhibitor compounds for treatment of inflammatory vascular diseases.

### BACKGROUND

The arachidonate cascade is a ubiquitous lipid signaling cascade that liberates arachidonic acid from the plasma membrane lipid reserves in response to a variety of extra-cellular and/or intra-cellular signals. The released arachidonic acid is then available to act as a substrate for a variety of oxidative enzymes that convert it to signaling lipids that have been implicated in inflammation and other diseases. Several commercially available drugs target and disrupt this pathway. Non-steroidal anti-inflammatory drugs (NSAIDs) disrupt the conversion of arachidonic acid to prostaglandins by inhibiting cyclooxygenases (COX1 and COX2). Asthma drugs, such as SINGULAIR™ or ACCOLATE block the effects of cysteinyl leukotrienes whereas Zileuton (Zyflo) disrupts the conversion of arachidonic acid to leukotrienes by inhibiting lipoxygenase (LOX).

Certain cytochrome P450-dependent enzymes convert arachidonic acid into a series of epoxide derivatives known as epoxyeicosatrienoic acids (EETs). These EETs are particularly prevalent in endothelium (cells that make up arteries and vascular beds), kidney, and lung. In contrast to many of the end products of the prostaglandin and leukotriene pathways, the EETs are reported to have a variety of anti-inflammatory and anti-hypertensive properties.

While EETs have potent effects in vivo, the epoxide moiety of the EETs is rapidly hydrolyzed into the less active dihydroxyeicosatrienoic acid (DHET) form by an enzyme called soluble epoxide hydrolase (sEH). Inhibition of sEH has been reported to significantly reduce blood pressure in hypertensive animals (see, e.g., Yu et al. Circ. Res. 87:992-8 (2000) and Sinal et al. J. Biol. Chem. 275:40504-10 (2000)), to reduce the production of proinflammatory nitric oxide (NO), cytokines, and lipid mediators, and to contribute to inflammatory resolution by enhancing lipoxin A₄ production in vivo (see Schmelzer et al. Proc. Nat'l Acad. Sci. USA 102(28):9772-7 (2005)). The sEH enzyme is coded by the EPXH2 gene.

### SUMMARY OF THE INVENTION

Disclosed herein are compositions and methods of using sEH inhibitory compounds for treatment of inflammatory vascular diseases. Examples of the inflammatory vascular diseases include, but are not limited to, in-stent stenosis, coronary arterial diseases (CAD), angina, acute myocardial infarction, acute coronary syndrome, chronic heart failure (CHF), peripheral arterial occlusive diseases (PAOD), critical limb ischemia (CLI), cardiac, kidney, liver and intestinal ischemia, renal failure, cardiac hypertrophy, etc. In some embodiments, the inflammatory vascular disease includes, but is not limited to, atherosclerosis, abdominal aortic aneurysm, vasculitis, and carotid artery stenosis. In some embodiments, the long term effect of atherosclerosis and/or vascular inflammation particularly cranial vascular inflammation is the significant increase in likelihood of stroke.

In one aspect, there is provided a method for treating inflammatory vascular disease in a subject, comprising administering to the subject an effective amount of a soluble epoxide hydrolase (sEH) inhibitor.

In some embodiments, the inflammatory vascular disease is selected from the group consisting of in-stent stenosis, coronary arterial disease, angina, acute myocardial infarction, acute coronary syndrome, chronic heart failure, peripheral arterial occlusive disease, critical limb ischemia, cardiac, kidney, liver or intestinal ischemia, renal failure, and cardiac hypertrophy.

In some embodiments, the inflammatory vascular disease is atherosclerosis.

In some embodiments, the inflammatory vascular disease is abdominal aortic aneurysm.

In some embodiments, the inflammatory vascular disease is vasculitis.

In some embodiments, the inflammatory vascular disease is carotid artery stenosis.

In some embodiments, the inflammatory vascular disease may be a prelude to a stroke. In some embodiments, there is provided a method of preventing strokes with a sEH inhibitor. It is contemplated that sEH inhibitors inhibit platelet aggregation in vivo complementing their use in preventing stocks. See Fitzpatrick, F. A., et al., Inhibition of Cyclooxygenase Activity and Platelet Aggregation by Epoxyeicosatrienoic Acids, J. Biol. Chem., 261(32):15334-15338 (1986); Krötz, F., et al., Membrane Potential-Dependent Inhibition of Platelet Adhesion to Endothelial Cells by Epoxyeicosatrienoic Acids, Arterioscler. Thromb. Vasc. Biol., 24:595-600 (2004); and Zhang, L., et al., 11,12-Epoxyeicosatrienoic Acid Activates the L-Arginine/Nitric Oxide Patway in Human Platelets, Mol. cell Biochem., 308:51-56 (2008), which are hereby incorporated by reference in their entirety.

The methods described herein include the administration of an effective amount of a sEH inhibitor which is a compound of Formula (I), Formula (II), Formula (III), or Formula (IV), or a stereoisomer, tautomer, or pharmaceutically acceptable salt thereof.

In some embodiments, the methods described herein include the administration of an effective amount of a sEH inhibitor which is a compound of Formula (I) or a stereoisomer, tautomer, or pharmaceutically acceptable salt thereof:

R¹LC(=Q)NHR² (I)

wherein:
Q is selected from the group consisting of O and S;
L is selected from the group consisting of a covalent bond, alkylene, O, S and NH; and
R¹ and R² independently are selected from the group consisting of substituted alkyl, aryl, substituted aryl, heteroaryl, substituted heteroaryl, cycloalkyl, substituted cycloalkyl, heterocycloalkyl, and substituted heterocycloalkyl.

In some embodiments, the methods described herein include the administration of an effective amount of a sEH inhibitor which is a compound of Formula (II) or a stereoisomer, tautomer, or pharmaceutically acceptable salt thereof:

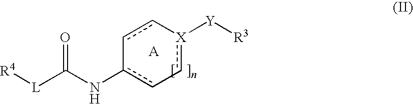

wherein:
L is selected from the group consisting of a covalent bond, alkylene, O, S and NH;
R³ is selected from the group consisting of alkyl, substituted alkyl, heteroaryl, substituted heteroaryl, heterocycloalkyl, and substituted heterocycloalkyl;
R⁴ is selected from the group consisting of aryl, substituted aryl, heteroaryl, substituted heteroaryl, cycloalkyl, substituted cycloalkyl, heterocycloalkyl, and substituted heterocycloalkyl;
n is 0, 1 or 2;
X is C, CH or N; provided that when X is C then n is 1 and ring A is phenyl; and
Y is selected from the group consisting of NH, O, C(═O)O, C(═O) and SO₂.

In one embodiment, X is N, n is 1 and ring A is piperidinyl.

In some embodiments, the methods described herein include the administration of an effective amount of a sEH inhibitor which is a compound of Formula (III) or a stereoisomer, tautomer, or pharmaceutically acceptable salt thereof:

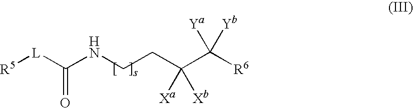

wherein:
L is selected from the group consisting of a covalent bond, alkylene, O, S and NH;
R⁵ is selected from the group consisting of aryl, substituted aryl, heteroaryl, substituted heteroaryl, cycloalkyl, substituted cycloalkyl, heterocycloalkyl, and substituted heterocycloalkyl;
s is 0-10;
R⁶ is selected from the group consisting of —CH₂OR⁷, —COR⁷, —COOR⁷, —CONR⁷R⁸, or a carboxylic acid isostere;
R⁷ and R⁸ independently are selected from the group consisting of hydrogen, alkyl, substituted alkyl, cycloalkyl, substituted cycloalkyl, heterocycloalkyl, substituted heterocycloalkyl, aryl, substituted aryl, heteroaryl, and substituted heteroaryl; or R⁷ and R⁸ together with the nitrogen atom bound thereto form a heterocycloalkyl ring having 3 to 9 ring atoms, and wherein said ring is optionally substituted with alkyl, substituted alkyl, heterocyclic, oxo or carboxy; and
each of X^{a}, X^{b}, Y^{a}, and Y^{b} independently is selected from the group consisting of hydrogen, C₁-C₄ alkyl, substituted C₁-C₄ alkyl, and halo, provided that at least one of Y^{a} and Y^{b} is halo or C₁-C₄ alkyl.

In some embodiments, the methods described herein include the administration of an effective amount of a sEH inhibitor which is a compound of Formula (IV) or a stereoisomer, tautomer, or pharmaceutically acceptable salt thereof:

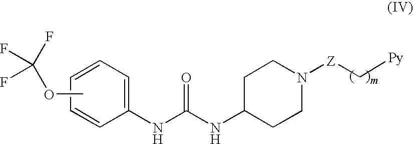

wherein
Z is CO or SO₂;
m is 0-2; and
Py is pyridyl or substituted pyridyl provided that when m is 0 then Z is on the 3- or 4-position of the pyridyl ring.

In some embodiments, the compound used in the methods provided herein, is selected from the group consisting of:
1-adamantyl-3-(1-(methylsulfonyl)piperidin-4-yl)urea;
1-(1-nicotinoylpiperidin-4-yl)-3-(4-(trifluoromethoxy)phenyl)urea;
1-adamantyl-3-(1-acetylpiperidin-4-yl)urea;
ethyl 2-fluoro-8-(3-adamantylureido)octanoate; and
2-fluoro-8-(3-adamantylureido)octanoic acid.

In another aspect, there is provided a method of treating a disease mediated at least in part by angiotensin (II) in a subject, comprising administering to the subject an effective amount of a sEH inhibitor.

In yet another aspect, there is provided a method of identifying a disease treatable by a sEH inhibitor in a diseased subject, wherein said method comprises:

a) identifying a diseased subject;

b) assaying a level of angiotensin II in said diseased subject to determine if said level is abnormal; and c) treating said diseased subject identified in b) above with abnormal level of angiotensin II with an sEH inhibitor.

In yet another aspect, there is provided a stent comprising a surface, wherein the surface comprises a biodegradable composition coating comprising an sEH inhibitor.

These and the other embodiments are further described in the text that follows.

### BRIEF DESCRIPTION OF THE DRAWINGS

This invention will be further described with reference being made to the accompanying drawings.

FIG. 1 illustrates that infusion of angiotensin II for 4 weeks induced abdominal aortic aneurysm (picture on left) in apolipoprotein E deficient mice, which can be partially prevented by the treatment with Compound 2 (picture on right).

FIG. 2 illustrates an average diameter of the suprarenal aorta in angiotensin II infused apoE deficient mice treated with Compound 2 and with vehicle.

FIG. 3 illustrates that infusion of angiotensin II for 4 weeks exacerbated the atherosclerotic lesion development in the carotid artery (picture on left) in apolipoprotein E deficient mice. Treatment with Compound 2 significantly reduced the lesion area (picture on right).

FIG. 4 illustrates that infusion of angiotensin II for 4 weeks exacerbated the atherosclerotic lesion development in the aortic arch (picture on left) in apolipoprotein E deficient mice. Treatment with Compound 2 significantly reduced the lesion area (picture on right).

FIG. 5 illustrates an atherosclerotic lesion area in the right carotid aretery in angiotensin II infused apoE deficient mice treated with Compound 2 and with vehicle (graph on the left); and an atherosclerotic lesion area in the aortic arch in angiotensin II infused apoE deficient mice treated with Compound 2 and with vehicle (graph on the right).

### DETAILED DESCRIPTION OF THE INVENTION

Throughout this disclosure, various publications, patents and published patent specifications are referenced by an identifying citation. The disclosures of these publications, patents and published patent specifications are hereby incorporated by reference in their entirety into the present disclosure to more fully describe the state of the art to which this invention pertains.

As used herein, certain terms have the following defined meanings.

As used in the specification and claims, the singular form “a”, “an” and “the” include plural references unless the context clearly dictates otherwise.

“C is-Epoxyeicosatrienoic acids” (“EETs”) are biomediators synthesized by cytochrome P450 epoxygenases.

“Epoxide hydrolases” (“EH;” EC 3.3.2.3) are enzymes in the alpha/beta hydrolase fold family that add water to 3 membered cyclic ethers termed epoxides.

“Soluble epoxide hydrolase” (“sEH”) is an enzyme which in cell converts EETs to dihydroxy derivatives called dihydroxyeicosatrienoic acids (“DHETs”). The cloning and sequence of the murine sEH is set forth in Grant et al., J. Biol. Chem. 268(23):17628-17633 (1993). The cloning, sequence, and accession numbers of the human sEH sequence are set forth in Beetham et al., Arch. Biochem. Biophys. 305(1):197-201 (1993). The evolution and nomenclature of the gene is discussed in Beetham et al., DNA Cell Biol. 14(1):61-71 (1995). Soluble epoxide hydrolase represents a single highly conserved gene product with over 90% homology between rodent and human (Arand et al., FEBS Lett., 338:251-256 (1994)).

“sEH inhibitor” refers to an inhibitor that inhibits by 50% the activity of sEH in hydrolyzing epoxides at a concentration of less than about 500 μM, preferably, the inhibitor inhibits by 50% the activity of sEH in hydrolyzing epoxides at a concentration of less than about 100 μM, even more preferably, the inhibitor inhibits by 50% the activity of sEH in hydrolyzing epoxides at a concentration of less than about 100 nM, and most preferably, the inhibitor inhibits by 50% the activity of sEH in hydrolyzing epoxides at a concentration of less than about 50 nM.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Alkenyl” refers to straight or branched hydrocarbyl groups having from 2 to 6 carbon atoms and preferably 2 to 4 carbon atoms and having at least 1 and preferably from 1 to 2 sites of vinyl (>C═C<) unsaturation. Such groups are exemplified, for example, by vinyl, allyl, and but-3-en-1-yl. Included within this term are the cis and trans isomers or mixtures of these isomers.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic (—C≡C—) unsaturation. Examples of such alkynyl groups include acetylenyl (—C≡CH), and propargyl (—CH₂C≡CH).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Alkylene” refers to a straight or branched, saturated or unsaturated, aliphatic, divalent radical. Alkylene includes methylene (—CH₂—), ethylene (—CH₂CH₂—), trimethylene (—CH₂CH₂CH₂—), tetramethylene (—CH₂CH₂CH₂CH₂—), 2-butenylene (—CH₂CH═CHCH₂—), 2-methyltetramethylene (—CH₂CH(CH₃—)CH₂CH₂—), pentamethylene (—CH₂CH₂CH₂CH₂CH₂—) and the like.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to a vinyl (unsaturated) carbon atom.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Acylamino” refers to the groups —NR²⁰C(O)alkyl, —NR²⁰C(O) substituted alkyl, —NR²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NR³¹R³² where R³¹ and R³² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R³¹ and R³² are optionally joined, together with the nitrogen bound thereto to form a heterocyclic or substituted heterocyclic group, provided that R³¹ and R³² are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R³¹ is hydrogen and R³² is alkyl, the substituted amino group is sometimes referred to herein as alkylamino. When R³¹ and R³² are alkyl, the substituted amino group is sometimes referred to herein as dialkylamino. When referring to a monosubstituted amino, it is meant that either R³¹ or R³² is hydrogen but not both. When referring to a disubstituted amino, it is meant that neither R³¹ nor R³² are hydrogen.

“Aminocarbonyl” refers to the group —C(O)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonyl” refers to the group —C(S)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR²⁰C(O)NR¹⁰R¹¹ where R²⁰ is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonylamino” refers to the group —NR²⁰C(S)NR¹⁰R¹¹ where R²⁰ is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyloxy” refers to the group —O—C(O)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyl” refers to the group —SO₂NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyloxy” refers to the group —O—SO₂NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonylamino” refers to the group —NR²⁰—SO₂NR¹⁰R¹¹ where R²⁰ is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amidino” refers to the group —C(═NR³²)NR¹⁰R¹¹ where R¹⁰R¹¹, and R³² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Carbonyl” refers to the divalent group —C(O)— which is equivalent to —C(═O)—.

“Isosteres” are different compounds that have different molecular formulae but exhibit the same or similar properties. For example, tetrazole is an isostere of carboxylic acid because it mimics the properties of carboxylic acid even though they both have very different molecular formulae. Tetrazole is one of many possible isosteric replacements for carboxylic acid. Other carboxylic acid isosteres contemplated by the present invention include —SO₃H, —SO₂NHR^{k′}, —PO₂(R^{k′})₂, —CN, —PO₃(R^{k′})₂, —OR^{k}, —SR^{k′}, —NHCOR^{k′}, —N(R^{k′})₂, —CONH(O)R^{k′}, —CONHNHSO₂R^{k′}, —COHNSO₂R^{k′}, —SO₂NHCOR^{k′}, —SO₂NHNHCOR^{k′}, and —CONR^{k′}CN, where R^{k′} is selected from hydrogen, hydroxyl, halo, haloalkyl, thiocarbonyl, alkoxy, alkenoxy, aryloxy, cyano, nitro, imino, alkylamino, aminoalkyl, thiol, thioalkyl, alkylthio, sulfonyl, alkyl, alkenyl, alkynyl, aryl, aralkyl (-(alkyl)-(aryl)), cycloalkyl, heteroaryl, heterocycle, and CO₂R^{m′} where R^{m′} is hydrogen, alkyl or alkenyl. In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of preferred carboxylic acid isosteres contemplated by this invention.

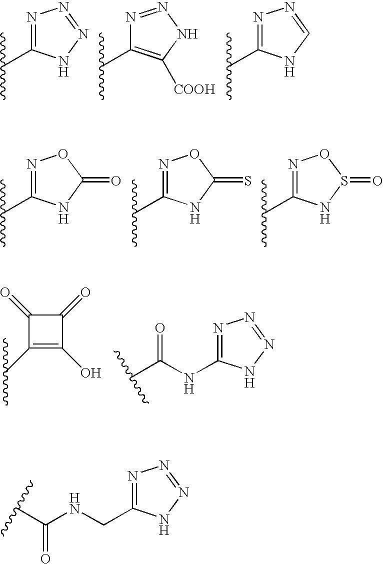

“Carboxy” or “carboxyl” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino” refers to the group —NR²⁰—C(O)O-alkyl, —NR²⁰—C(O)O— substituted alkyl, —NR²⁰—C(O)O-alkenyl, —NR²⁰—C(O)O-substituted alkenyl, —NR²⁰—C(O)O-alkynyl, —NR²⁰—C(O)O-substituted alkynyl, —NR²⁰—C(O)O-aryl, —NR²⁰—C(O)O-substituted aryl, —NR²⁰—C(O)β-cycloalkyl, —NR²⁰—C(O)O-substituted cycloalkyl, —NR²⁰—C(O)β-cycloalkenyl, —NR²⁰—C(O)O-substituted cycloalkenyl, —NR²⁰—C(O)O-heteroaryl, —NR²⁰—C(O)O-substituted heteroaryl, —NR²⁰—C(O)O-heterocyclic, and —NR—C(O)O-substituted heterocyclic wherein R²⁰ is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)β-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” refers to the group —CN.

“Cycloalkyl” refers to cyclic alkyl groups of from 3 to 10 carbon atoms having single or multiple cyclic rings including fused, bridged, and spiro ring systems. One or more of the rings can be aryl, heteroaryl, or heterocyclic provided that the point of attachment is through the non-aromatic, non-heterocyclic ring carbocyclic ring. Examples of suitable cycloalkyl groups include, for instance, adamantyl, cyclopropyl, cyclobutyl, cyclopentyl, and cyclooctyl. Other examples of cycloalkyl groups include bicycle[2,2,2,]octanyl, norbornyl, and spiro groups such as spiro[4.5]dec-8-yl:

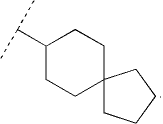

“Cycloalkenyl” refers to non-aromatic cyclic alkyl groups of from 3 to 10 carbon atoms having single or multiple cyclic rings and having at least one >C═C<ring unsaturation and preferably from 1 to 2 sites of >C═C<ring unsaturation.

“Substituted cycloalkyl” and “substituted cycloalkenyl” refers to a cycloalkyl or cycloalkenyl group having from 1 to 5 or preferably 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Cycloalkyloxy” refers to —O-cycloalkyl.

“Substituted cycloalkyloxy” refers to —O-(substituted cycloalkyl).

“Cycloalkylthio” refers to —S-cycloalkyl.

“Substituted cycloalkylthio” refers to —S-(substituted cycloalkyl).

“Cycloalkenyloxy” refers to —O-cycloalkenyl.

“Substituted cycloalkenyloxy” refers to —O-(substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to NR²³C(═NR²³)N(R²³)₂ where each R²³ is independently selected from the group consisting of hydrogen, alkyl, substituted alkyl, aryl, substituted aryl, heteroaryl, substituted heteroaryl, heterocyclic, and substituted heterocyclic and two R²³ groups attached to a common guanidino nitrogen atom are optionally joined together with the nitrogen bound thereto to form a heterocyclic or substituted heterocyclic group, provided that at least one R²³ is not hydrogen, and wherein said substituents are as defined herein.

“Halo” or “halogen” refers to fluoro, chloro, bromo and iodo and preferably is fluoro or chloro.

“Haloalkyl” refers to alkyl groups substituted with 1 to 5, 1 to 3, or 1 to 2 halo groups, wherein alkyl and halo are as defined herein.

“Haloalkoxy” refers to alkoxy groups substituted with 1 to 5, 1 to 3, or 1 to 2 halo groups, wherein alkoxy and halo are as defined herein.

“Haloalkylthio” refers to alkylthio groups substituted with 1 to 5, 1 to 3, or 1 to 2 halo groups, wherein alkylthio and halo are as defined herein.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl. In some embodiments, the substituted heteroaryl is substituted pyridyl. The substituted pyridyl is within the meaning of the scope as set forth above.

“Heteroaryloxy” refers to —O-heteroaryl.

“Substituted heteroaryloxy” refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Heterocycle encompasses single ring or multiple condensed rings, including fused bridged and spiro ring systems. In fused ring systems, one or more the rings can be cycloalkyl, aryl, or heteroaryl provided that the point of attachment is through the non-aromatic ring. In one embodiment, the nitrogen and/or sulfur atom(s) of the heterocyclic group are optionally oxidized to provide for the N-oxide, sulfinyl, or sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocyclyl.

“Substituted heterocyclyloxy” refers to the group —O-(substituted heterocyclyl).

“Heterocyclylthio” refers to the group —S-heterocyclyl.

“Substituted heterocyclylthio” refers to the group —S-(substituted heterocyclyl).

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O) or (—O⁻).

“Spiro ring systems” refers to bicyclic ring systems that have a single ring carbon atom common to both rings.

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Substituted sulfonyl includes groups such as methyl-SO₂—, phenyl-SO₂—, and 4-methylphenyl-SO₂—. The term “alkylsulfonyl” refers to —SO₂-alkyl. The term “(substituted sulfonyl)amino” refers to —NH(substituted sulfonyl) wherein substituted sulfonyl is as defined herein.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cylcoalkyl, —OSO₂-cycloalkenyl, —OSO₂-substituted cylcoalkenyl, —OSO₂-aryl, —OSO₂-substituted aryl, —OSO₂-hetero aryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thiol” refers to the group —SH.

“Thiocarbonyl” refers to the divalent group —C(S)— which is equivalent to —C(═S)—.

“Thione” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

“Stereoisomer” or “stereoisomers” refer to compounds that differ in the chirality at one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate.

“Pharmaceutical composition” is intended to include the combination of an active agent with a carrier, inert or active, making the composition suitable for diagnostic or therapeutic use in vitro, in vivo or ex vivo.

“Pharmaceutically-acceptable carrier” encompasses any of the standard pharmaceutical carriers, such as a phosphate-buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton (1975)).

An “excipient” refers to an inert substance added to a pharmaceutical composition to further facilitate administration of the active ingredient.

A “subject,” “individual” or “patient” is used interchangeably herein, and refers to a vertebrate, for example a mammal or preferably a human. Mammals include, but are not limited to, murines, rats, simians, humans, farm animals, sport animals and pets.

A “sample,” as used herein, means a material known to or suspected of expressing a level of angiotensin II. The test sample can be used directly as obtained from the source or following a pretreatment to modify the character of the sample. The sample can be derived from any biological source, such as tissues or extracts, including cells, and physiological fluids, such as, for example, whole blood, plasma, serum, ocular lens fluid, cerebrospinal fluid, synovial fluid, peritoneal fluid and the like. The sample is obtained from animals or humans, preferably from humans. The sample can be treated prior to use, such as preparing plasma from blood, diluting viscous fluids, and the like. Methods of treating a sample can involve filtration, distillation, extraction, concentration, inactivation of interfering components, the addition of reagents, and the like.

An “effective amount” is used synonymously with a “therapeutically effective amount” and intends an amount sufficient to effect beneficial or desired results. An effective amount can be administered in one or more administrations, applications, or dosages.

“Treating” or “treatment” of a disease, disorder or condition will depend on the disease, disorder or condition to be treated and the individual to be treated. In general, treatment intends one or more of (1) inhibiting the progression of the manifested disease, disorder or condition as measured by clinical or sub-clinical parameters (where the term “inhibiting” or “inhibition” is intended to be a subset of “treating” or “treatment”), (2) arresting the development of the disease, disorder or condition as measured by clinical or sub-clinical parameters, (3) ameliorating or causing regression of the disease, disorder or condition as measured by clinical or sub-clinical parameters, or (4) reducing pain or discomfort for the subject as measured by clinical parameters. “Treating” does not include preventing the onset of the disease or condition.

“Preventing” or “prevention” of a disease, disorder or condition means that the onset of the disease or condition in a subject predisposed thereto is prevented such that subject does not manifest the disease, disorder or condition.

### A. Methods

Disclosed herein are methods for treating inflammatory vascular disease in a subject, comprising administering to the subject an effective amount of a sEH inhibitor. The inflammatory vascular disease includes, but is not limited to, in-stent stenosis, coronary arterial diseases (CAD), angina, acute myocardial infarction, acute coronary syndrome, chronic heart failure (CHF), peripheral arterial occlusive diseases (PAOD), critical limb ischemia (CLI), cardiac, kidney, liver and intestinal ischemia, renal failure, cardiac hypertrophy, etc. In some embodiments, the inflammatory vascular disease includes, but is not limited to, atherosclerosis, abdominal aortic aneurysm, vasculitis, and carotid artery stenosis. In some embodiments, the vascular inflammation and atherosclerosis may lead to stroke.

In some embodiments, there is provided a method for treating atherosclerosis in a subject, comprising administering to the subject an effective amount of a sEH inhibitor. Atherosclerosis is a chronic inflammatory disease of the arterial wall characterized by progressive accumulation of lipids, cells (macrophages, lymphocytes, and smooth muscle cells), and extracellular matrix proteins. Inflammatory cells, which are present in arterial lesions, can be players in various processes such as plaque progression, plaque rupture, and vessel thrombosis. Chronic exposure to low-density lipoprotein (LDL) modified by oxidation or enzymatic attack can activate endothelial cells and cells in the underlying intima to express adhesion molecules and inflammatory genes that promote monocyte accumulation and macrophage differentiation in developing atherosclerotic plaques. Pattern recognition receptors can play a role in this innate immune response that leads to local inflammation and both innate and adaptive immune responses. Diseases such as, type 2 diabetes may be associated with significantly accelerated rates of macrovascular complications such as atherosclerosis.

In some embodiments, there is provided a method for treating abdominal aortic aneurysm in a subject, comprising administering to the subject an effective amount of a sEH inhibitor. Abdominal aeortic aneurysm (AAA) is the condition when the aeortic artery leading from the heart distends. AAA can be inflammatory abdominal aortic aneurysm (AAA) or atherosclerotic AAA. Both inflammatory and atherosclerotic AAA may affect the infrarenal portion of the abdominal aorta. Patients with the inflammatory variant may be younger and symptomatic, such as back or abdominal pain. Inflammatory AAA may have an elevated erythrocyte sedimentation rate or abnormalities of other serum inflammatory markers. The inflammatory variant may be characterized pathologically by marked thickening of the aneurysm wall, an extraordinary expansion of the adventitia due to inflammation, fibrosis of the adjacent retroperitoneum, and rigid adherence of the adjacent structures to the anterior aneurysm wall.

In some embodiments, there is provided a method for treating vasculitis in a subject, comprising administering to the subject an effective amount of a sEH inhibitor. Vasculitis is an inflammation of the blood vessels in the body. In vasculitis, the body's immune system may mistakenly attack the body's own blood vessels, causing them to become inflamed. Inflammation can damage the blood vessels and lead to a number of serious complications. For example, when a blood vessel becomes inflamed, it may narrow, making it more difficult for blood to get through; close off completely so that blood can't get through at all (occlusion); and/or stretch and weaken so much that it bulges (aneurysm) and may possibly burst (aneurysm rupture). The disruption in blood flow from inflammation can damage the body's organs. Specific signs and symptoms depend on which organ has been damaged and the extent of the damage. It has previously been shown that sEH inhibitors can reduce hypertension. See e.g. U.S. Pat. No. 6,531,506.

In some embodiments, there is provided a method for treating carotid artery stenosis in a subject, comprising administering to the subject an effective amount of a sEH inhibitor. Carotid stenosis is a narrowing of the lumen of the carotid artery, which may be caused by atherosclerosis. The carotid stenosis may be the stenosis in the proximal part of the internal carotid artery (at the carotid bulb). Stenosis in other parts of the carotid arteries may also occur. Atherosclerotic carotid stenosis may be asymptomatic or it may cause symptoms by embolism to either cerebral vessels in the brain or to the retinal arteries. Emboli to the cerebral arteries can cause transient ischaemic attack (TIA) or cerebrovascular accident (CVA). Emboli to the retina can produce amaurosis fugax or retinal infarction.

In some embodiments, there is provided a method for inhibiting stroke in a subject, comprising administering to the subject an effective amount of a sEH inhibitor. Stroke can be caused by extracranial atherosclerotic disease of the carotid arteries and aortic arch vessels, and in such embodiments, the patient is first selected to be at risk for stroke by evaluation of the extent of atherosclerotic deposits and/or inflammation in the carotid arteries.

In another aspect, there is provided a method of treating a disease mediated at least in part by angiotensin (II) in a subject, comprising administering to the subject an effective amount of a soluble epoxide hydrolase (sEH) inhibitor.

Angiotensin II (Ang II) is a pro-inflammatory factor. Ang II can promote vascular inflammation, accelerate atherosclerosis, and induce abdominal aeortic aneurysm. Ang II can induce a variety of vascular events including endothelial activation and dysfunction, cell proliferation, and monocyte chemoattraction, which can contribute to atherosclerosis development. Induction of macrophage cholesterol biosynthesis and macrophage uptake of modified lipoproteins can be additional mechanisms contributing to the atherogenic action of Ang II. The effect of ACE inhibitor on Ang II to prevent atherosclerosis and vascular inflammation induced by Ang II (Cunha et al. Atherosclerosis 178:9-17 (2005)) and the effect of IFN-13 on Ang II (Zhang et al. Atherosclerosis 197:204-211 (2008)) have been reported.

The sEH inhibitor compounds of the invention can be used to attenuate the effect of Ang II by enhancing the effect of EETs which have anti-hypertensive and anti-inflammatory effects. In some embodiments, there is provided a method of treating atherosclerosis mediated at least in part by angiotensin (II) in a subject, comprising administering to the subject an effective amount of a soluble epoxide hydrolase (sEH) inhibitor.

Ang II has been implicated in inducing abdominal aeortic aneurysm (Wang et al. Circulation 111:2219-2226 (2005); Martin-McNulty et al. Arterioscler Thromb Vasc Biol. 23:1627-1632 (2003); Deng et al. Circ Res. 92:510-517 (2003)). In some embodiments, there is provided a method of treating abdominal aeortic aneurysm mediated at least in part by angiotensin (II) in a subject, comprising administering to the subject an effective amount of a soluble epoxide hydrolase (sEH) inhibitor.

In yet another aspect, there are provided methods for diagnostic assay to identify a disease in a subject treatable by a sEH inhibitor and to identify the subjects that would benefit from the therapeutic methods of the invention.

In some embodiments, there is provided a method of identifying a disease treatable by a sEH inhibitor in a diseased subject, wherein the method comprises:

a) identifying a diseased subject;

b) assaying a level of angiotensin II in the diseased subject to determine if the level is abnormal; and

c) treating the diseased subject identified in b) above with abnormal level of angiotensin II with an sEH inhibitor.

The abnormal level of angiotensin II in a subject includes a level of angiotensin II that is higher or lower than normal. As provided supra, angiotensin II is a pro-inflammatory factor and can promote vascular inflammation, accelerate atherosclerosis, and induce abdominal aeortic aneurysm. The determination of the level of angiotensin II in a subject or in a sample of the subject can lead to the identification of the disease that can be treated by the sEH inhibitor compounds of the invention.

Assays for determining the level of angiotensin II in the subject are well known in the art. Some of the examples of the assays are described in Simon et al. Clinical Chemistry 38:1963-1967 (1992); Barrett et al. Journal of Pharmacology And Experimental Therapeutics, 170(2):326-333 (1969); and Nussberger et al. International Journal of Environmental Analytical Chemistry 25(1):257-268 (1986).

The identification of a level of angiotensin II may involve one or more comparisons with reference samples. The reference samples may be obtained from the same subject or from a different subject who is either not affected with the disease (such as, normal subject) or is a patient. The reference sample could be obtained from one subject, multiple subjects or is synthetically generated. The identification may also involve the comparison of the identification data with the databases. In some embodiments, the step of correlating the level of angiotensin II of subjects with normal subjects is performed by a software algorithm.

The identification and analysis of the level of angiotensin II can help in, for example, distinguishing disease states to inform prognosis, selection of therapy of treatment with sEH inhibitors and/or prediction of therapeutic response, disease staging, prediction of efficacy of treatment with sEH inhibitor, prediction of adverse response with treatment, and detection of recurrence.

The determination of the level of angiotensin II and the subsequent identification of a disease in a subject treatable by sEH inhibitors, as disclosed herein, can be used to enable or assist in the pharmaceutical drug development process for sEH inhibitor compounds. The determination of the level of angiotensin II can be used to diagnose disease for patients enrolling in a clinical trial. The determination of the level of angiotensin II can indicate the state of the disease of patients undergoing treatment in clinical trials, and show changes in the state during the treatment with sEH inhibitors. The determination of the level of angiotensin II can demonstrate the efficacy of treatment with sEH inhibitors, and can be used to stratify patients according to their responses to various therapies.

In some embodiments, patients, health care providers, such as doctors and nurses, or health care managers, use the level of angiotensin II in a subject to make a diagnosis or prognosis and select treatment options with sEH inhibitors. In some embodiments, the methods described herein can be used to predict the likelihood of response for any individual to a treatment with sEH inhibitors, select a treatment with sEH inhibitor, or to preempt any adverse effects of treatments on a particular individual. Also, the methods can be used to evaluate the efficacy of treatments over time.

For example, samples can be obtained from a patient over a period of time as the patient is undergoing treatment with sEH inhibitor. The level of angiotensin II in the different samples can be compared to each other to determine the efficacy of the treatment. The samples from a subject can be collected repeatedly over a longitudinal period of time (e.g., about once a day, once a week, once a month, biannually or annually). Obtaining numerous samples from a subject over a period of time can be used to verify results from earlier detections and/or to identify an alteration in biological pattern as a result of, for example, disease progression, treatment with sEH inhibitor, etc. Also, the methods described herein can be used to compare the efficacy of the therapies and/or responses to one or more treatments in different populations (e.g., ethnicities, family histories, etc.).

In some embodiments, the sEH inhibitor compound is used in combination with another therapeutic agent. Combination therapy includes administration of a single pharmaceutical dosage formulation which contains a sEH inhibitor and one or more additional active agents, or therapies such as heat, light and such, as well as administration of the sEH inhibitor and each active agent in its own separate pharmaceutical dosage formulation. For example, a compound of this invention and one or more of other agents including, but not limited to, COX2 inhibitors, PDE5 inhibitors angiotensin concerting enzyme inhibitors, and angiotensin II receptor blockers, could be administered to the human subject together in a single oral dosage composition such as a tablet or capsule or each agent can be administered in separate oral dosage formulations. Combination therapy is understood to include all these regimens.

In some embodiments, there is provided a stent comprising a surface, wherein the surface comprises a biodegradable composition coating comprising an sHE inhibitor. In some embodiments, the biodegradable composition is a polymer. This stent can be implanted in a subject suffering from a disease mediated at least in part by angiotensin II. The stent can be coated with one or more of the sEH inhibitors as provided herein.

sEH inhibitors are contemplated to inhibit platelet aggregation in vivo.

### B. sEH Inhibitory Compounds

In the methods provided herein, an effective amount of a sEH inhibitor, or composition comprising a sEH inhibitor, is administered to a subject in need thereof. sEH inhibitors are well known in the art and include but are not limited to those disclosed in McElroy et al, J. Med. Chem., 46:1066-1080 (2003); U.S. Pat. Nos. 6,831,082, and 6,693,130, US Patent Application Publications 2007/0225283, 2006/0270609, 2008/0076770, 2008/0032978, 2008/153889, 2008/0207621, 2008/0207622, 2008/0200444, 2008/0200467, 2008/0227780, 2009/0023731, 2009/0082395, 2009/0082350, 2009/0082456 and 2009/0082423, U.S. patent application Ser. No. 12/426,136, and International patent applications WO2008/105968, WO2007/043652, WO2007/043653, WO2007/106705, WO2007/067836, WO2007/098352, WO2008/022171, WO2006/121719, WO2007/044491, WO2006/121684, WO2009/020960 and PCT/US2008/088244. All of the above listed publications, patents, patent applications are incorporated by reference in their entirety. For example the sEH inhibitors are compounds described by at least one of the following general or specific formulas shown in Formula (I), Formula (II), Formula (III), or Formula (IV), or in Tables 1 and 2.

In one aspect, the compound is of Formula (I) or a stereoisomer, tautomer, or pharmaceutically acceptable salt thereof:

R¹LC(=Q)NHR² (I)

wherein:
Q is selected from the group consisting of O and S;
L is selected from the group consisting of a covalent bond, alkylene, O, S and NH; and
R¹ and R² are independently selected from the group consisting of substituted alkyl, aryl, substituted aryl, heteroaryl, substituted heteroaryl, cycloalkyl, substituted cycloalkyl, heterocycloalkyl, and substituted heterocycloalkyl.

In some embodiments, L is NH.

In some embodiments, R¹ is cycloalkyl, substituted cycloalkyl, phenyl or substituted phenyl. In some embodiments, R² is substituted alkyl or substituted heterocycloalkyl. In some embodiments, R² is substituted phenyl.

In some embodiments, Q is O.

In some embodiments, the compound is of Formula (II) or a stereoisomer, tautomer, or pharmaceutically acceptable salt thereof:

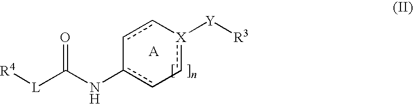

wherein:
L is selected from the group consisting of a covalent bond, alkylene, O, S and NH;
R³ is selected from the group consisting of alkyl, substituted alkyl, heteroaryl, substituted heteroaryl, heterocycloalkyl, and substituted heterocycloalkyl;
R⁴ is selected from the group consisting of aryl, substituted aryl, heteroaryl, substituted heteroaryl, cycloalkyl, substituted cycloalkyl, heterocycloalkyl, and substituted heterocycloalkyl;
n is 0, 1 or 2;
X is C, CH or N; provided that when X is C then n is 1 and ring A is phenyl; and
Y is selected from the group consisting of NH, O, C(═O)O, C(═O) and SO₂.

In one embodiment, X is N, n is 1 and ring A is piperidinyl.

In some embodiments, R⁴ is adamantyl or substituted adamantyl.

In some embodiments, R⁴ is phenyl. In some embodiments, R⁴ is substituted phenyl.

In some embodiments, Y is C(═O). In some embodiments, Y is SO₂. In some embodiments, Y is C(═O)O. In some embodiments, Y is O. In some embodiments, Y is NH.

In some embodiments, R³ is alkyl or substituted alkyl.

In some embodiments, the compound is of Formula (III), or a stereoisomer, tautomer, or pharmaceutically acceptable salt thereof:

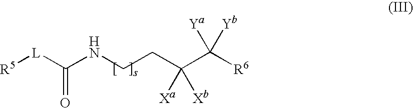

wherein:
L is selected from the group consisting of a covalent bond, alkylene, O, S and NH;
R⁵ is selected from the group consisting of aryl, substituted aryl, heteroaryl, substituted heteroaryl, cycloalkyl, substituted cycloalkyl, heterocycloalkyl, and substituted heterocycloalkyl;
s is 0-10;
R⁶ is selected from the group consisting of —OR⁷, —CH₂OR⁷, —COR⁷, —COOR⁷, —CONR⁷R⁸, or a carboxylic acid isostere;
R⁷ and R⁸ independently are selected from the group consisting of hydrogen, alkyl, substituted alkyl, cycloalkyl, substituted cycloalkyl, heterocycloalkyl, substituted heterocycloalkyl, aryl, substituted aryl, heteroaryl, and substituted heteroaryl; or R⁷ and R⁸ together with the nitrogen atom bound thereto form a heterocycloalkyl ring having 3 to 9 ring atoms, and wherein said ring is optionally substituted with alkyl, substituted alkyl, heterocyclic, oxo or carboxy; and
each of X^{a}, X^{b}, Y^{a}, and Y^{b} is independently selected from the group consisting of hydrogen, C₁-C₄ alkyl, substituted C₁-C₄ alkyl, and halo.

In some embodiments, R⁵ is adamantyl or substituted adamantyl. In some embodiments, R⁵ is phenyl. In some embodiments, R⁵ is substituted phenyl.

In some embodiments, R⁶ is selected from the group consisting of —CH₂OR⁷, —COR⁷, —COOR⁷, —CONR⁷R⁸, or a carboxylic acid isostere.

In some embodiments, at least one of Y^{a} and Y^{b} is halo or C₁-C₄ alkyl. In some embodiments, at least one of Y^{a} and Y^{b} is halo.

In some embodiments, the compound is of Formula (IV), or a stereoisomer, tautomer, or pharmaceutically acceptable salt thereof:

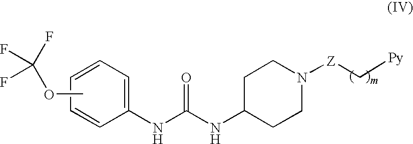

wherein
Z is CO or SO₂;
m is 0-2; and
Py is pyridyl or substituted pyridyl provided that when m is 0 then Z is on the 3- or 4-position of the pyridyl ring.

In some embodiments, Z is CO.

In some embodiments, m is 0.

In some embodiments, m is 1.

In some embodiments, m is 0 and Z is on the 3-position of the pyridyl ring.

In some embodiments, the compound is a compound, a stereoisomer, tautomer, or pharmaceutically acceptable salt thereof selected from Table 1 or 2.

TABLE 1

| Compound | |
|---|---|
| No. | Name |
| 1 | 1-adamantyl-3-(1-(methylsulfonyl)piperidin-4-yl)urea |
| 2 | 1-(1-nicotinoylpiperidin-4-yl)-3- |
| | (4(trifluoromethoxy)phenyl)urea |
| 3 | 1-adamantyl-3-(1-acetylpiperidin-4-yl)urea |
| 4 | ethyl 2-fluoro-8-(3-adamantylureido)octanoate |
| 5 | 2-fluoro-8-(3-adamantylureido)octanoic acid |

TABLE 2

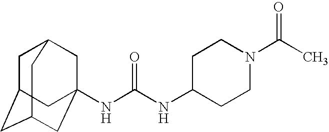

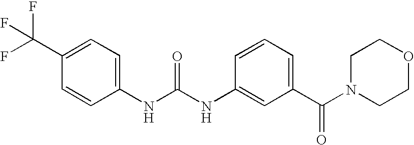

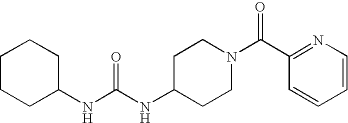

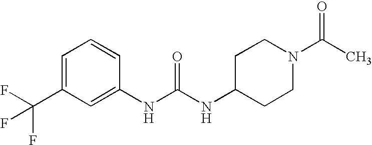

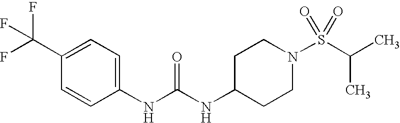

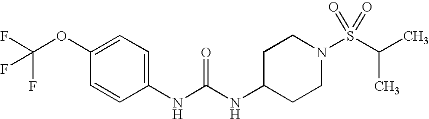

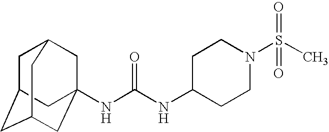

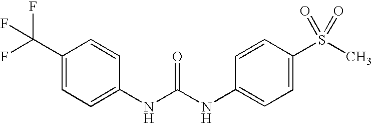

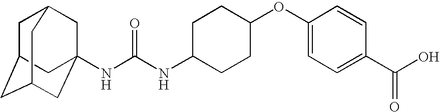

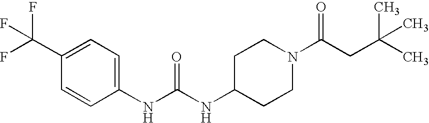

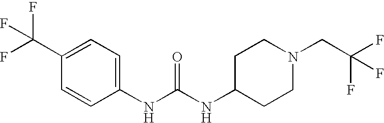

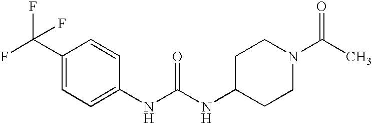

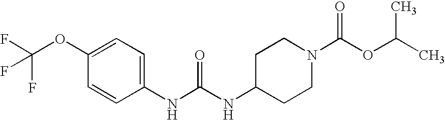

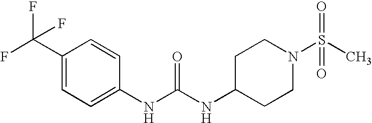

For the purpose of clarity, the compounds listed above can be referred to by their compound number or an alternative name. For example, 1-adamantyl-3-(1-(methylsulfonyl)piperidin-4-yl)urea can be referred to as Compound 1 or, alternatively, 1-[1-(methylsulfonyl)piperidin-4-yl]-N′-(adamant-1-yl) urea. Likewise, 1-adamantyl-3-(1-acetylpiperidin-4-yl)urea can be referred to as Compound 3 or, alternatively, N-(1-acetylpiperidin-4-yl)-N′-(adamant-1-yl)urea.

In some embodiments, the compound used in the methods provided herein is 1-adamantyl-3-(1-(methylsulfonyl)piperidin-4-yl)urea.

In some embodiments, the compound is 1-(1-nicotinoylpiperidin-4-yl)-3-(4-(trifluoromethoxy)phenyl)urea.

In some embodiments, the compound is 1-adamantyl-3-(1-acetylpiperidin-4-yl)urea.

In some embodiments, the compound is ethyl 2-fluoro-8-(3-adamantylureido)octanoate.

In some embodiments, the compound is 2-fluoro-8-(3-adamantylureido)octanoic acid.

In another aspect, one or more of the compounds of Formula (I), (II), (III), or (IV) or a stereoisomer, tautomer, or pharmaceutically acceptable salt thereof, may be used in the preparation of a medicament for the treatment of an inflammatory vascular disease, as provided herein.

### C. Compositions and Formulations

The compositions are comprised of, in general, a sEH inhibitor in combination with at least one pharmaceutically acceptable carrier or excipient. Acceptable carriers are known in the art. Acceptable carriers or excipients are non-toxic, aid administration, and do not adversely affect the therapeutic benefit of the compound. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols.

The sEH inhibitors can be administered in any suitable formulation such as a tablet, pill, capsule, semisolid, gel, transdermal patch or solution, powders, sustained release formulation, solution, suspension, elixir or aerosol. The most suitable formulation will be determined by the disease or disorder to be treated and the individual to be treated.

Compressed gases may be used to disperse a sEH inhibitor of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The following are representative pharmaceutical formulations containing a sEH inhibitor of the present invention.

### Tablet Formulation

The following ingredients are mixed intimately and pressed into single scored tablets.

| Ingredient | Quantity per tablet, mg |
|---|---|
| sEH inhibitor | 400 |
| Cornstarch | 50 |
| Croscarmellose sodium | 25 |
| Lactose | 120 |
| Magnesium stearate | 5 |

### Capsule Formulation

The following ingredients are mixed intimately and loaded into a hard-shell gelatin capsule.

| Ingredient | Quantity per capsule, mg |
|---|---|
| sEH inhibitor | 200 |
| Lactose, spray-dried | 148 |
| Magnesium stearate | 2 |

### Suspension Formulation

The following ingredients are mixed to form a suspension for oral administration (q.s.=sufficient amount).

| Ingredient | Amount | |
|---|---|---|
| sEH inhibitor | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.0 | g |
| Sorbitol (70% solution) | 13.0 | g |
| Veegum K (Vanderbilt Co) | 1.0 | g |
| Flavoring | 0.035 | mL |
| colorings | 0.5 | mg |

| | |
|---|---|
| distilled water | q.s. to 100 mL |

### Injectable Formulation

The following ingredients are mixed to form an injectable formulation.

| Ingredient | Quantity per injection, mg |
|---|---|
| sEH inhibitor | 0.2 mg-20 mg |
| sodium acetate buffer solution, 0.4 M | 2.0 mL |
| HCl (1N) or NaOH (1N) | q.s. to suitable pH |
| water (distilled, sterile) | q.s. to 20 mL |

### Suppository Formulation

A suppository of total weight 2.5 g is prepared by mixing the compound of the invention with Witepsol® H-15 (triglycerides of saturated vegetable fatty acid; Riches-Nelson, Inc., New York), and has the following composition:

| Ingredient | Quantity per suppository, mg |
|---|---|
| sEH inhibitor | 500 mg |
| Witepsol ® H-15 | Balance |

Also provided is a medicament comprising a compound or composition as described herein for use in treating a disease or disorder as described above, which can be identified by noting any one or more clinical or sub-clinical parameters.

### D. Dosing and Administration

The present invention provides therapeutic methods generally involving administering to a subject in need thereof an effective amount of sEH inhibitors described herein. The dose, frequency, and timing of such administering will depend in large part on the selected therapeutic agent, the nature of the condition to be treated, the condition of the subject, including age, weight and presence of other conditions or disorders, the formulation of the therapeutic agent and the discretion of the attending physician. The sEH inhibitors and compositions described herein and the pharmaceutically acceptable salts thereof are administered via oral, parenteral, subcutaneous, intramuscular, intravenous or topical routes. Generally, it is contemplated that the sEH inhibitors are to be administered in dosages ranging from about 0.10 milligrams (mg) up to about 1000 mg per day, although variations will necessarily occur, depending, as noted above, on the target tissue, the subject, and the route of administration. In preferred embodiments, the sEH inhibitors are administered orally once or twice a day.

The sEH inhibitors are preferably administered in a range between about 0.10 mg and 1000 mg per day, more preferably the compounds are administered in a range between about 1 mg and 800 mg per day; more preferably, the compounds are administered in a range between about 2 mg and 600 mg per day; more preferably, the compounds are administered in a range between about 5 mg and 500 mg per day; yet more preferably, the compounds are administered in a range between about 10 mg and 200 mg per day; yet even more preferably, the compounds are administered in a range between about 50 mg and 100 mg per day.

The following examples are provided to illustrate certain aspects of the present invention and to aid those of skill in the art in practicing the invention. These examples are in no way to be considered to limit the scope of the invention.

### E. Synthetic Chemistry

The sEH inhibitors of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the sEH inhibitors of this invention may contain one or more chiral centers. Accordingly, if desired, such inhibitors can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Preferably, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4^{th} Edition), and Larock's Comprehensive Organic Transformations (VCH Publishers Inc., 1989).

The various starting materials, intermediates, and compounds of the invention may be isolated and purified where appropriate using conventional techniques such as precipitation, filtration, crystallization, evaporation, distillation, and chromatography. Characterization of these compounds may be performed using conventional methods such as by melting point, mass spectrum, nuclear magnetic resonance, and various other spectroscopic analyses.

Scheme 1 below illustrates a general synthetic method for the preparation of the compounds of Formula (I).

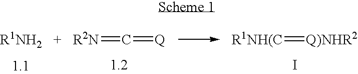

A synthesis of the compounds of the invention is shown in Scheme 1, where Q, R¹, and R² are as defined herein. Specifically, amine 1.1 reacts with the appropriate isocyanate or thioisocyanate 1.2 to form the corresponding urea or thiourea of Formula (I). Typically, the formation of the urea is conducted using a polar solvent such as DMF (dimethylformamide) at 0 to 10° C. Isocyanate or thioisocyanate 1.2 can be either known compounds or can be prepared from known compounds by conventional synthetic procedures. Suitable isocyanates include by way of example only, adamantyl isocyanate, cyclohexyl isocyanate, phenyl isocyanate, trifluoromethylphenyl isocyanate, chlorophenyl isocyanate, fluorophenyl isocyanate, trifluoromethoxyphenyl isocyanate and the like.

Scheme 2 illustrates the methods of Scheme 1 as they relate to the preparation of piperidinyl compounds of Formula (II).

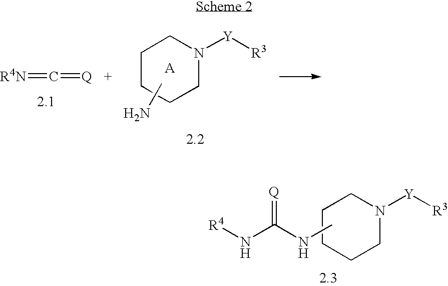

Scheme 2 can also be employed for the synthesis of compounds of Formula (II) where, for illustrative purposes, ring A is a piperidinyl ring and Q, Y, R³, and R⁴ are as defined herein. Reaction of 2.1 with amine 2.2 forms the corresponding urea or thiourea 2.3.

In Scheme 2, the N—(YR³) substituted piperidinyl amine can be prepared as shown in Scheme 3 below:

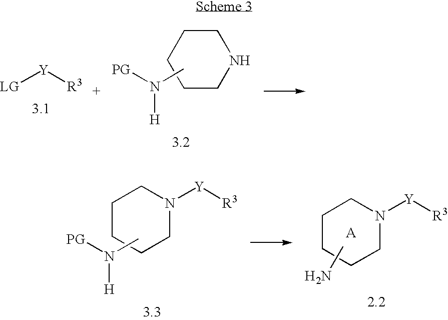

Y and R³ are as defined herein; LG is a leaving group such as a halo group, a tosyl group, a mesyl group, and the like; and PG is a conventional amino protecting group such as a tert-butoxycarbonyl (Boc) group. Reaction of 3.1 with protected aminopiperidine 3.2 forms the functionalized amine 3.3. Removal of the protecting group gives 2.2. Both of these reactions are well known in the art.

The following Schemes 4-7 illustrate preferred methods of preparing compounds of Formula (I) and/or (II). Specifically, in Scheme 4, a 4-amidopiperidine group is employed for illustrative purposes only and this scheme illustrates the synthesis of N-(1-acylpiperidin-4-yl)-N′-(adamant-1-yl)urea compounds where R³ is as defined herein:

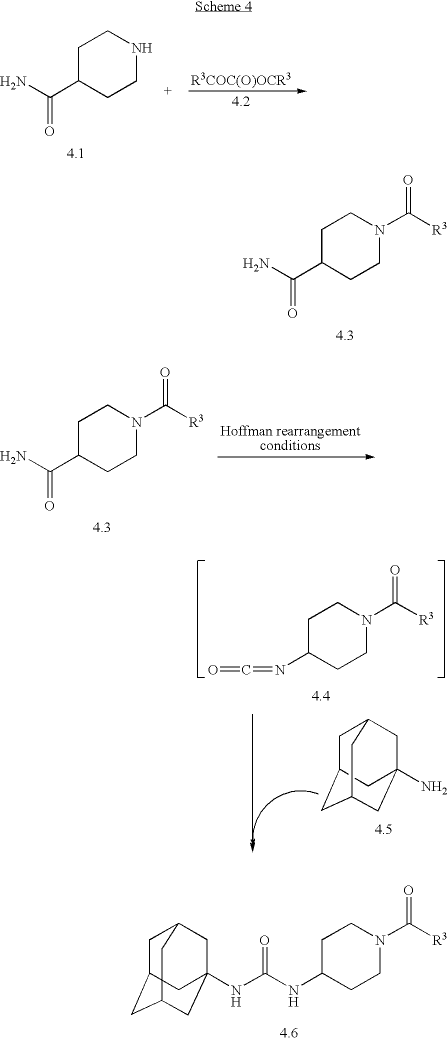

In Scheme 4, the amino group of compound 4.1 is acylated using conventional conditions. Specifically, a stoichiometric equivalent or slight excess of a carboxylic acid anhydride 4.2 (which is used only for illustrative purposes) is reacted with compound 4.1 in the presence of a suitable inert diluent such as tetrahydrofuran, chloroform, methylene chloride and the like. When an acid chloride is employed in place of the acid anhydride, the reaction is typically conducted in the presence of an excess of a suitable base to scavenge the acid generated during the reaction. Suitable bases are well known in the art and include, by way of example only, triethylamine, diisopropylethylamine, pyridine, and the like.

The reaction is typically conducted at a temperature of from about 0 to about 40° C. for a period of time sufficient to effect substantial completion of the reaction which typically occurs within about 1 to about 24 hours. Upon reaction completion, the acylpiperidylamide, compound 4.3, can be isolated by conventional conditions such as precipitation, evaporation, chromatography, crystallization, and the like or, alternatively, used in the next step without isolation and/or purification. In certain cases, compound 4.3 precipitates from the reaction.

Compound 4.3 is then subjected to Hoffman rearrangement conditions to form isocyanate compound 4.4 under conventional conditions. In certain cases, Hoffman rearrangement conditions comprise reacting with an oxidative agent preferably selected from (diacetoxyiodo)benzene, base/bromine, base/chlorine, base/hypobromide, or base/hypochloride. Specifically, approximately stoichiometric equivalents of the N-acyl-4-amidopiperidine, compound 4.3, and, e.g., (diacetoxyiodo)benzene are combined in the presence of a suitable inert diluent such as acetonitrile, chloroform, and the like. The reaction is typically conducted at a temperature of from about 40° C., to about 100° C., and preferably at a temperature of from about 70° C., to about 85° C., for a period of time sufficient to effect substantial completion of the reaction which typically occurs within about 0.1 to about 12 hours. Upon reaction completion, the intermediate isocyanate, compound 4.4, can be isolated by conventional conditions such as precipitation, evaporation, chromatography, crystallization, and the like.

Alternatively and preferably, this reaction is conducted in the presence of adamantyl amine, compound 4.5, such that upon formation of the isocyanate, compound 4.4, the isocyanate functionality of this compound can react in situ with the amino functionality of compound 4.5 to provide for compound 4.6. In this embodiment, the calculated amount of the intermediate isocyanate is preferably employed in excess relative to the adamantyl amine and typically in an amount of from about 1.1 to about 1.2 equivalents based on the number of equivalents of adamantyl amine employed. The reaction conditions are the same as set forth above and the resulting product can be isolated by conventional conditions such as precipitation, evaporation, chromatography, crystallization, and the like.

Compound 4.4 is a stable intermediate. In certain cases, compound 4.4 is formed substantially free from impurities.

Scheme 5 below illustrates an alternative synthesis of a urea compound where a 4-amidopiperidine is employed for illustrative purposes:

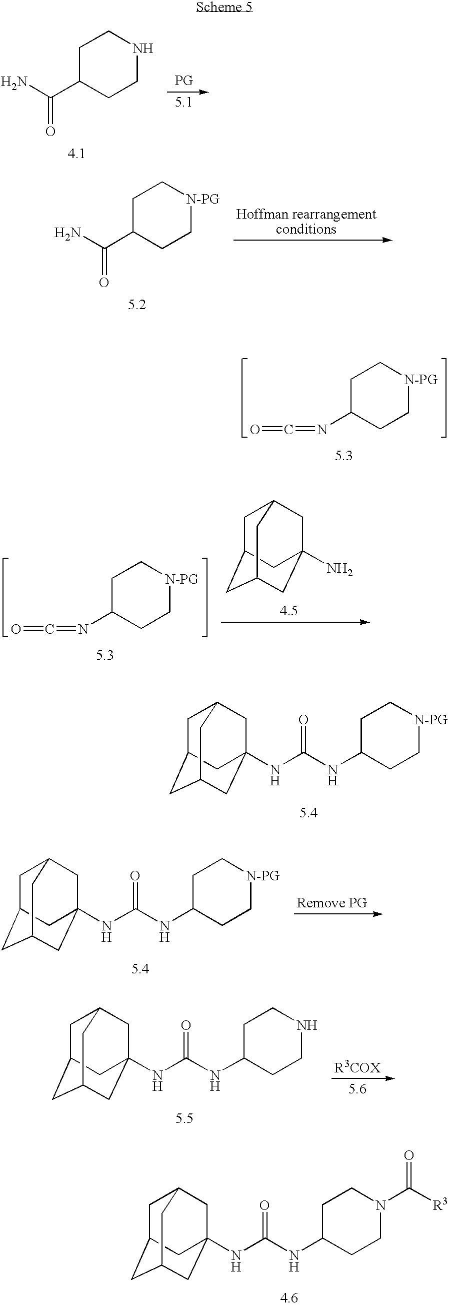

where R³ and PG are as defined herein and X is selected from the group consisting of OH, halo and OC(O)R³.

Specifically, in Scheme 5, coupling of the adamantyl urea to the piperidinyl ring occurs prior to acylation of the piperidinyl nitrogen atom. In Scheme 5, the amine functionality of compound 4.1 is protected using a conventional amino protecting group (PG) which is well known in the art. In certain cases, the amino protecting group is a benzyl protecting group which can be derived from benzyl chloride and benzyl bromide. Compound 5.2 is subjected to Hoffman rearrangement conditions to form isocyanate compound 5.3 in the manner described in detail above. Compound 5.3 is a stable intermediate. The reaction of compound 5.3 with adamantyl amine 4.5 is conducted as provided in Scheme 4. The reaction is preferably conducted in a single reaction step wherein intermediate compound 5.3 is reacted in situ with adamantyl amine 4.5, to form compound 5.4. Compound 5.4 is subjected to conditions to remove the protecting group to yield compound 5.5. In certain cases, the protecting group is benzyl and the removal conditions employ palladium-carbon with methanol and formic acid. Compound 5.5 is acylated with compound 5.6 to form compound 4.6.

Scheme 6 below illustrates the synthesis of N-(1-alkylsulfonylpiperidin-4-yl)-N′-(adamant-1-yl)ureas:

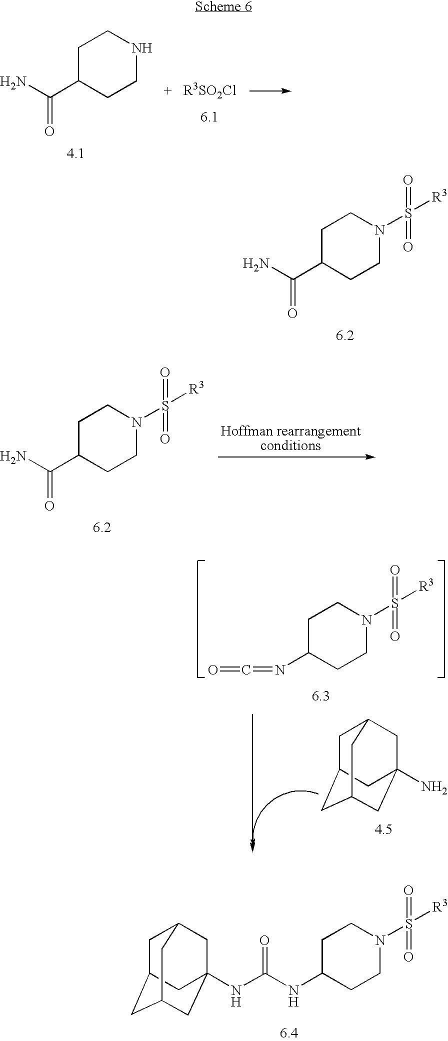

wherein R³ is defined herein.

Specifically, in Scheme 6, amino compound 4.1 is reacted with a sulfonyl halide 6.1 (used for illustrative purposes only), to provide for sulfonamide compound 6.2. This reaction is typically conducted by reacting the amino compound 4.1 with at least one equivalent, preferably about 1.1 to about 2 equivalents, of the sulfonyl halide (for illustrative purposes depicted as the sulfonyl chloride) in an inert diluent such as dichloromethane, chloroform and the like. Generally, the reaction is preferably conducted at a temperature ranging from about −10° C. to about 20° C. for about 1 to about 24 hours. Preferably, this reaction is conducted in the presence of a suitable base to scavenge the acid generated during the reaction. Suitable bases include, by way of example, tertiary amines, such as triethylamine, diisopropylethylamine, N-methylmorpholine and the like. Alternatively, the reaction can be conducted under Schotten-Baumann-type conditions using aqueous alkali, such as sodium hydroxide and the like, as the base. Upon completion of the reaction, the resulting sulfonamide, compound 6.2, is recovered by conventional methods including neutralization, extraction, precipitation, chromatography, filtration, and the like or, alternatively, used in the next step without purification and/or isolation.

Compound 6.2 is subjected to Hoffman rearrangement conditions as described above to form isocyanate compound 6.3. The reaction of compound 6.3 with adamantyl amine 4.5, is conducted as provided in Scheme 4 and is preferably conducted in a single reaction step wherein the isocyanate compound 6.3, is reacted in situ with adamantyl amine 4.5 to form compound 6.4.

The sulfonyl chlorides employed in the above reaction are also either known compounds or compounds that can be prepared from known compounds by conventional synthetic procedures. Such compounds are typically prepared from the corresponding sulfonic acid, using phosphorous trichloride and phosphorous pentachloride. This reaction is generally conducted by contacting the sulfonic acid with about 2 to 5 molar equivalents of phosphorous trichloride and phosphorous pentachloride, either neat or in an inert solvent, such as dichloromethane, at temperature in the range of about 0° C. to about 80° C. for about 1 to about 48 hours to afford the sulfonyl chloride. Alternatively, the sulfonyl chloride can be prepared from the corresponding thiol compound, i.e., from compounds of the formula R³—SH where R³ is as defined herein, by treating the thiol with chlorine (Cl₂) and water under conventional reaction conditions.

Compound 6.3 is a stable intermediate. In certain cases, compound 6.3 is formed substantially free from impurities.

Scheme 7 below illustrates an alternative synthesis of a urea compound.

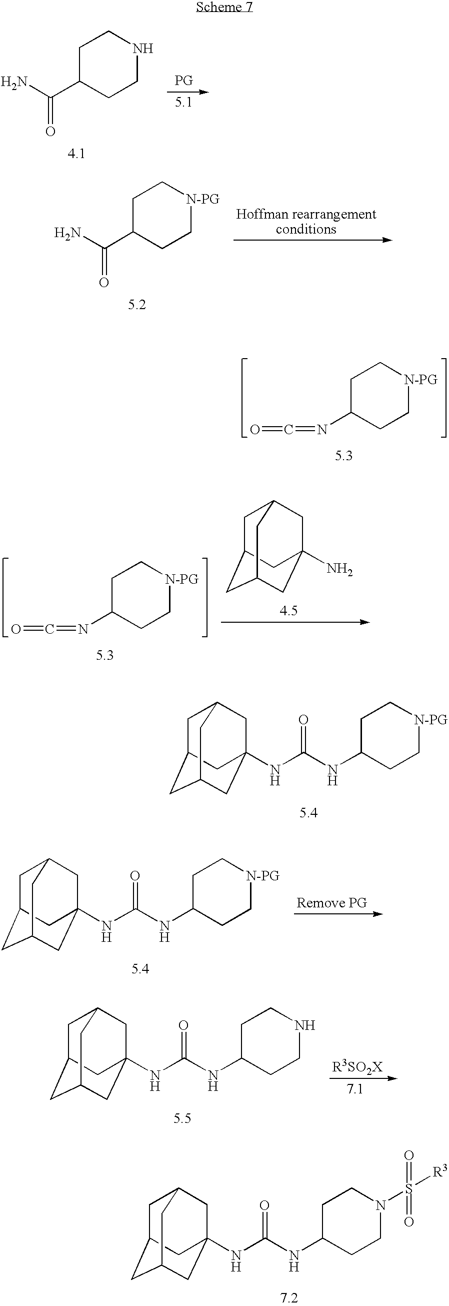

wherein R³ and PG are as defined herein and X is selected from the group consisting of OH, halo and —OC(O)R³.

Specifically, in Scheme 7, coupling of the adamantyl urea, compound 4.5, to the piperidinyl ring occurs prior to sulfonylation of the piperidinyl nitrogen atom. In Scheme 7, the amine functionality of compound 4.1 is protected using a conventional amino protecting group (PG) which are well known in the art. In certain cases, the amino protecting group is a benzyl protecting group which can be derived from benzyl chloride or benzyl bromide. Compound 5.2 is subjected to Hoffman rearrangement conditions to form isocyanate compound 5.3 in the manner described in detail above. Compound 5.3 is a stable intermediate. The reaction of compound 5.3 with adamantyl amine 4.5, is conducted as provided in Scheme 4 and is preferably conducted in a single reaction step wherein intermediate compound 5.3 is reacted in situ with adamantyl amine 4.5, to form compound 5.4. Compound 5.4 is subjected to conditions to remove the protecting group to yield compound 5.5. In certain cases, the protecting group is benzyl and the removal conditions employ palladium-carbon with methanol and formic acid. Compound 5.5 is then sulfonylated with compound 7.1 to form compound 7.2 as per Scheme 6 above.

The following schemes 8-10 illustrate preferred methods of preparing compounds of Formula (I) and/or (III) represented by compound 8.3 (Scheme 8).

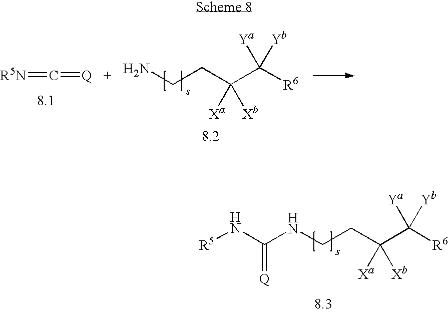

Specifically, as depicted in Scheme 9, synthesis of ethyl amino-2-fluoroalk-2-enoate 9.6 is shown for illustrative purposes only:

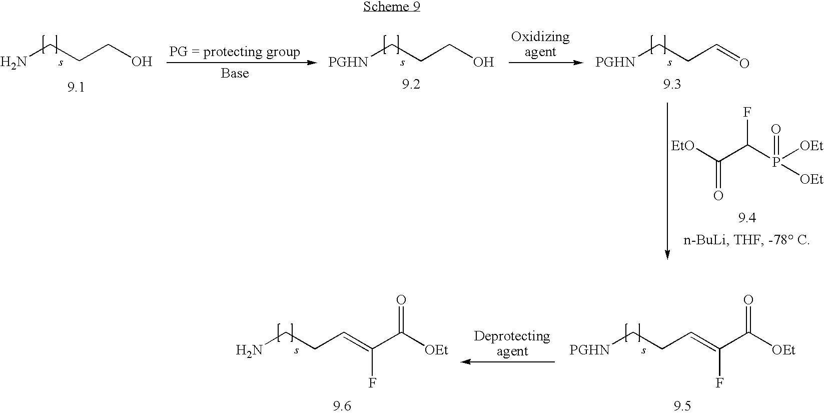

In Scheme 9, s is as defined herein. The synthesis of the compounds of the invention can be exemplified by, but is not limited to, the preparation of the intermediate 9.6, as shown in Scheme 9. Amine 9.1 can be protected with any amine protecting group known in the art (for example, 2,4-dimethoxy-benzyl (DMB), tert-butoxycarbonyl (Boc) etc.) to give compounds 9.2. For example, amine 9.1 can be treated with t-Boc anhydride in the presence of a base, such as sodium carbonate, and a suitable solvent such as, THF to give compounds 9.2. Upon reaction completion, 9.2 can be recovered by conventional techniques such as neutralization, extraction, precipitation, chromatography, filtration and the like; or, alternatively, used in the next step without purification and/or isolation.

Compounds 9.2 are then treated with any suitable oxidizing agent known in the art, to give aldehydes 9.3. For example, 9.2 can be treated with pyridinium chlorochromate (PCC) and neutral alumina (Al₂O₃) in the presence of a suitable solvent, such as, dichloromethane (DCM) to give 9.3. Upon reaction completion, 9.3 can be recovered by conventional techniques such as neutralization, extraction, precipitation, chromatography, filtration and the like; or, alternatively, used in the next step without purification and/or isolation.

Compounds 9.3 are then treated with triethyl-2-fluoro-2-phosphonoacetate 9.4 to give compounds 9.5. This is typically performed in dry tetrahydrofuran (THF) or another suitable solvent known to one skilled in the art, typically at, but not limited to, room temperature in the presence of n-butyllithium (n-BuLi), or another suitable base known to one skilled in the art. Upon reaction completion, 9.5 can be recovered by conventional techniques such as neutralization, extraction, precipitation, chromatography, filtration and the like; or, alternatively, used in the next step without purification and/or isolation.

Compounds 9.5 are then deprotected using a suitable deprotecting agent known in the art to give the intermediate 9.6. For example, deprotection can be achieved, in addition to other methods known to one skilled in the art, by treatment of 9.5 with SOCl₂ in a suitable solvent such as dichloromethane (DCM) (preferred method for PG=2,4-dimethoxy-benzyl (DMB)). Alternatively, 9.5 can be deprotected with TFA neat or in a suitable solvent known to one skilled in the art such as, DCM to give the compounds 9.6 (preferred method for PG=tert-butoxycarbonyl (Boc)). Upon reaction completion, 9.6 can be recovered by conventional techniques such as neutralization, extraction, precipitation, chromatography, filtration and the like; or, alternatively, used in the next step without purification and/or isolation.

The synthesis of the compounds of the invention can be exemplified by, but is not limited to, the use of the intermediate 9.6 to prepare the compounds of the invention, as shown in Scheme 10.

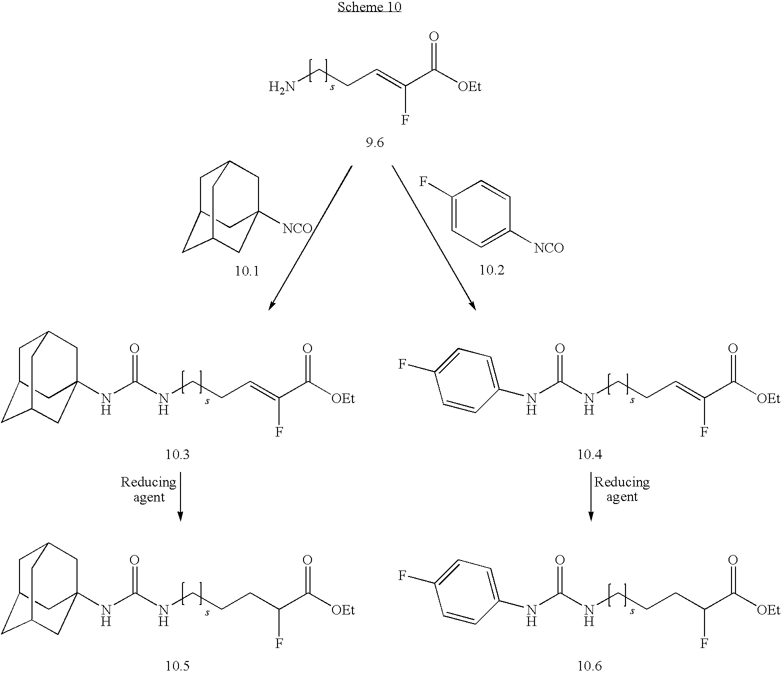

The intermediate 9.6 can be treated with appropriate isocyanate compounds 10.1 or 10.2 to form the corresponding adamantyl compounds 10.3 or phenyl compounds 10.4. Without limiting the scope of the present invention, Scheme 10 shows p-fluorophenyl or unsubstituted adamantyl for illustration purposes only. Any suitably substituted or unsubstituted phenyl or adamantyl can be used in Scheme 10 to yield the compounds of the invention. Typically, the reaction with isocyanates is conducted using DCM in the presence of triethylamine (TEA) at room temperature, or alternatively, a polar solvent such as DMF (dimethylformamide) at 0 to 10° C. Isocyanate compounds 10.1 or 10.2 can be either known compounds or compounds that can be prepared from known compounds by conventional synthetic procedures. Upon reaction completion, 10.3 and/or 10.4 can be recovered by conventional techniques such as neutralization, extraction, precipitation, chromatography, filtration and the like; or, alternatively, used in the next step without purification and/or isolation.

Compounds 10.3 or 10.4 can then be reduced using any suitable reducing agent known in the art, to give compounds 10.5 or 10.6, respectively. For example, 10.3 or 10.4 can be hydrogenated with palladium/carbon (Pd/C) in the presence of a suitable solvent known in the art such as, methanol, at suitable temperature such as, room temperature. Upon reaction completion, 10.5 and/or 10.6 can be recovered by conventional techniques such as neutralization, extraction, precipitation, chromatography, filtration and the like. Alternatively, the ester group of the adamantyl compounds 10.3 or phenyl compounds 10.4 can be hydrolyzed (not shown in scheme 10) to give the corresponding acid compounds. The hydrolysis of esters is well known in the art. For example, the ester can be hydrolyzed using lithium hydroxide (LiOH) in the presence of a suitable solvent such as, but not limited to THF/methanol/water. The resulting acids can then be reduced with reducing agents as described above to give the corresponding adamantyl or phenyl compounds of the invention.

The following schemes 11-13 illustrate preferred methods of preparing compounds of Formula (I) and/or (IV).

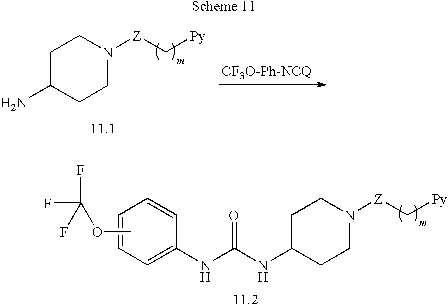

A synthesis of the compounds of the invention, in particular compounds of Formula IV, is shown in Scheme 11, where Z, m, and Py are as defined herein. Reaction of 11.1 with trifluorophenylisocyanate or trifluorophenylisothiocyanate gives the corresponding urea or thiourea 11.2. Typically, the preparation of the urea is conducted using a polar solvent such as DMF (dimethylformamide) at 60 to 85° C. Generally, amine 11.1 may be readily available from commercial sources or prepared by conventional methods and procedures known to a person of skill in the art.

Alternatively, compounds of Formula I or IV may be prepared according to Scheme 12 from compounds 12.1 wherein Pr is an amino protecting group, such as tert-butoxycarbonyl (Boc), benzyloxycarbonyl (Cbz), and 9-fluorenylmethyloxycarbonyl (Fmoc) and m, Z, and Py are as defined herein. Compounds 12.1 may be prepared using a method similar to Scheme 11.

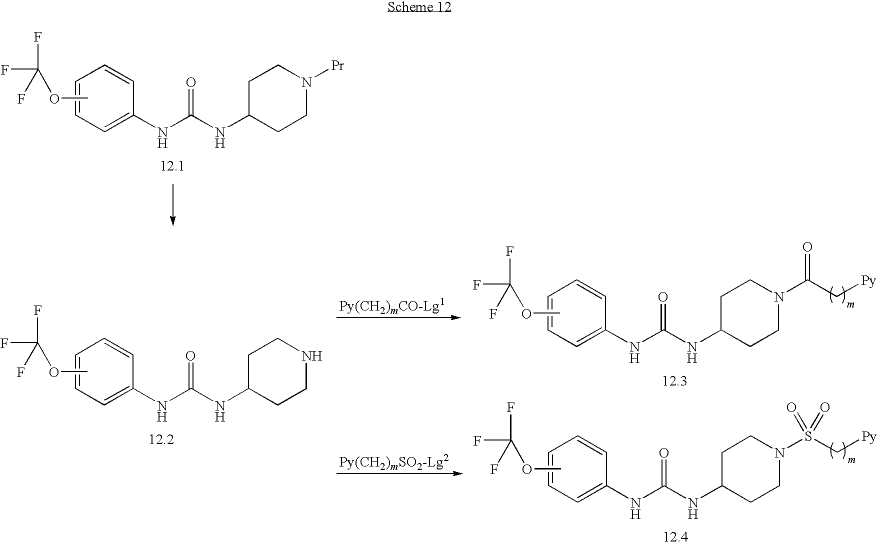

As shown in Scheme 12, Compound 12.1 can be deprotected to the free amino compound 12.2 under conditions known for deprotecting the particular protecting group used. For example, when Pr is Boc, it can be removed under acidic conditions using an acid, such as HCl or trifluoroacetic acid; when Pr is Cbz, it can be removed under hydrogenation conditions, such as using hydrogen gas in the presence of a catalyst, such as palladium on carbon; when Pr is Fmoc, it can be removed under basic conditions using a base such as piperidine. Compound 12.2 can then react with Py-(CH₂)ₘ—CO-Lg¹ (Lg¹ is OH or a leaving group such as halo) to form the amide compounds 12.3 or react with Py-(CH₂)ₘSO₂-Lg² (Lg² is a leaving group such as halo) to form the sulfonamide compounds 12.4. The reaction conditions for these reactions are well known to a person of skill in the art.

The urea compounds of this invention can also be prepared according to Scheme 13 where Z, m, and py are defined herein and Lg is a suitable leaving group.

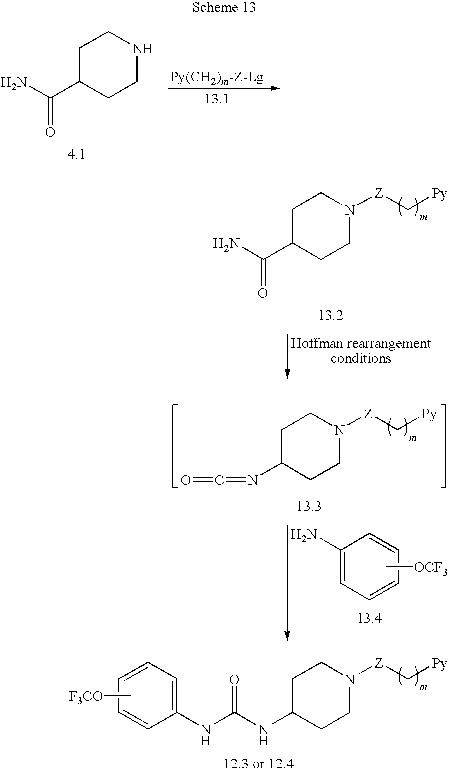

In Scheme 13, the amino group of compound 4.1 reacts with Py(CH₂)ₘ—Z-Lg 13.1 (LG is OH or a leaving group such as halo) to form the corresponding amide or sulfonamide 13.2.

Compound 13.2 is then subjected to Hoffman rearrangement conditions to form isocyanate compound 13.3 under conventional conditions. In certain cases, Hoffman rearrangement conditions comprise reacting with an oxidative agent preferably selected from (diacetoxyiodo)benzene, base/bromine, base/chlorine, base/hypobromide, or base/hypochloride. Specifically, approximately stoichiometric equivalents of compound 13.2, and, e.g., (diacetoxyiodo)benzene are combined in the presence of a suitable inert diluent such as acetonitrile, chloroform, and the like. The reaction is typically conducted at a temperature of from about 40° C., to about 100° C., and preferably at a temperature of from about 70° C., to about 85° C., for a period of time sufficient to effect substantial completion of the reaction which typically occurs within about 0.1 to about 12 hours. Upon reaction completion, the intermediate isocyanate compound 13.3 can be isolated by conventional conditions such as precipitation, evaporation, chromatography, crystallization, and the like.

Alternatively and preferably, this reaction is conducted in the presence of trifluoromethoxyphenyl amine 13.4, such that upon formation of the isocyanate 13.3, the isocyanate functionality of this compound can react in situ with the amino functionality to provide for compound 12.3 or 12.4 depending on Z. In this embodiment, the calculated amount of the intermediate isocyanate is preferably employed in excess relative to the amine and typically in an amount of from about 1.1 to about 1.2 equivalents based on the number of equivalents of the amine employed. The reaction conditions are the same as set forth above and the resulting product can be isolated by conventional conditions such as precipitation, evaporation, chromatography, crystallization, and the like.

Compound 13.3 is a stable intermediate. In certain cases, compound 13.3 is formed substantially free from impurities.

A further elaboration of processes suitable for preparing compounds of Formula (I), Formula (II), Formula (III), and Formula (IV), are disclosed in Anandan et al., U.S. Provisional Application Ser. No. 61/017,380, filed on Dec. 28, 2007; Hammock et al., International Application No. PCT/US2007/006412, filed on Mar. 13, 2007; and, Gless et al., U.S. Provisional Application Ser. No. 61/046,316, filed on Apr. 18, 2008, all of which are herein incorporated by reference in their entirety.

The following examples are provided to illustrate certain aspects of the present invention and to aid those of skill in the art in practicing the invention. These examples are in no way to be considered to limit the scope of the invention.

### EXAMPLES

The examples below as well as throughout the application, the following abbreviations have the following meanings. If not defined, the terms have their generally accepted meanings.

| | |
|---|---|
| aq. = | aqueous |
| bd = | broad doublet |
| bs = | broad singlet |
| bt = | broad triplet |
| Boc = | tert-butoxycarbonyl |
| BuLi = | butyl lithium |
| CH₂Cl₂ = | dichloromethane |
| d = | doublet |
| DCM = | dichloromethane |
| DMF = | dimethylformamide |
| Et₃N = | triethylamine |
| EtOAc = | ethylacetate |
| g = | gram |
| h = | hour(s) |
| HCl = | hydrochloric acid |
| Kg = | kilogram |
| LiOH = | lithium hydroxide |
| m = | multiplet |
| mg = | milligram |
| MeOH = | methanol |
| MHz = | megahertz |
| mL = | milliliter |
| mM = | millimolar |
| mmol = | millimole |
| m.p. = | melting point |
| MS = | mass spectroscopy |
| NaHCO₃ = | sodium bicarbonate |
| Na₂SO₄ = | sodium sulfate |
| NMR = | nuclear magnetic resonance |
| Pd/C = | palladium over carbon |
| q = | quartet |
| s = | singlet |
| t = | triplet |
| THF = | tetrahydrofuran |
| TFA = | trifluoroacetic acid |
| TLC = | thin layer chromatography |

### Example 1

### Synthesis of 1-Adamantyl-3-(1-(methylsulfonyl)piperidin-4-yl)urea (1)

A reactor was charged with 1.0 mole-equivalent of 4-piperidinecarboxamide, 16.4 mole-equivalents of THF, and 1.2 mole-equivalents of N,N-(diisopropyl)ethylamine under a nitrogen atmosphere. The resulting mixture was cooled to 0-5° C. internal, and 1.2 mole-equivalents of methanesulfonyl chloride was added at such a rate as to maintain an internal temperature of less than 10° C. After addition was complete, the reaction mixture was stirred allowing the temperature to rise to 20° C. internal. The reaction contents was monitored until the amount of unreacted 4-piperidinecarboxamide was less than 1% relative to N-methanesulfonyl piperid-4-yl amide product (typically about 2-12 hours). The precipitated product was collected by filtration then washed with dichloromethane to remove excess (diisopropyl)ethylamine hydrochloride. The solid product was dried to constant weight in a vacuum oven under a nitrogen bleed maintaining an internal temperature of 50° C. to afford product as a light yellow solid in 87% yield. ¹H NMR (DMSO-d₆): 7.30 (s, 1H), 6.91 (s, 1H), 3.46-3.59 (m, 2H), 2.83 (s, 3H), 2.60-2.76 (m, 2H), 2.08-2.24 (m, 1H), 1.70-1.86 (m, 2H), 1.43-1.62 (m, 2H); MS: 207 [M+H]⁺; m.p. 126-128° C.

A reactor was charged with 1.00 mole-equivalents of N-methanesulfonyl piperid-4-yl amide, 1.06 mole-equivalents of 1-adamantyl amine, and 39.3 mole-equivalents of acetonitrile, and the resulting mixture was heated to 40° C. internal under a nitrogen atmosphere. (Diacetoxyiodo)benzene (1.20 mole-equivalents) was charged portionwise in such a way that the reaction mixture was maintained below 75° C. internal. After the (diacetoxyiodo)benzene had been added, the reaction mixture was heated at 65-70° C. internal, and the reaction contents monitored until the amount of unreacted 1-adamantyl amine was less than 5% relative to product N-(1-methanesulfonyl piperidin-4-yl)-N'-(adamant-1-yl)urea (typically less than about 6 hours). The resulting mixture was cooled to 20° C. internal and filtered to remove a small amount of insoluble material. The filtrate was allowed to stand for 48 hours at which point the precipitated product was collected by filtration. The solid product was dried to constant weight in a vacuum oven under a nitrogen bleed maintaining an internal temperature of 50° C. to afford product in 58% yield based on N-methanesulfonyl piperid-4-yl amide. ¹H NMR (CDCl₃): 3.95-4.08 (m, 2H), 3.74-3.82 (m, 2H), 3.63-3.82 (m, 1H), 3.78 (s, 3H), 3.70-3.80 (m, 2H), 2.02-2.12 (m, 5H), 1.90 (s, 6H), 1.67 (s, 6H), 1.40-1.50 (m, 2H); MS: 356 [M+H]⁺; m.p. 228-229° C.

### Example 2

### Synthesis of 1-(1-nicotinoylpiperidin-4-yl)-3-(4-(trifluoromethoxy)phenyl)urea (2)

To a solution of 4-amino-1-BOC-piperidine (22.0 g, 0.11 mol) in 300 mL CH₂Cl₂ was added 20.3 g (0.1 mol) p-trifluoromethoxyphenyl isocyanate at room temperature. The resulting clear solution was stirred for 18 h at room temperature, and the solvent was removed in vacuo. The resulting crude product was dissolved in MeOH (200 mL) and 137 mL (0.55 mol) 4.0 M aq. HCl in dioxane was added at room temperature. The resulting clear solution was stirred for 18 h at room temperature, and the solvent was removed in vacuo. The residue was dissolved in water (200 mL) and washed with EtOAc (2×100 mL). The water layer was basified to pH around 8 with saturated NaHCO₃ solution and extracted with EtOAc (2×150 mL). The combined organic extracts from the extraction of the basic solution were washed with water (100 mL) and brine (100 mL), and dried over Na₂SO₄. After removal of solvent, finally under high vacuum for 24 h, 1-(p-trifluoromethoxyphenyl)-3-(4-aminopiperidine)-urea was obtained as a white solid (24.8 g, 81%).

To a solution of 1-(ptrifluoromethoxyphenyl)-3-(4-aminopiperidine)-urea (909 mg, 3.0 mmol) in 30 mL CH₂Cl₂ was added sequentially 1.3 mL (9.0 mmol) Et₃N and 963 mg (4.5 mmol) nicotinoyl chloride hydrochloride salt with ice water cooling. The resulting mixture was stirred for 18 h at room temperature. The mixture was then diluted with water (30 mL) and CH₂Cl₂ (50 mL). The layers were phase separated, and the organic layer was washed with sat. NaHCO₃ solution (30 mL), water (30 mL), dried over Na₂SO₄, filtered, and concentrated in vacuo to afford crude product. Purification on a silica gel column eluting with 4% MeOH in CH₂Cl₂ afforded pure 1-(1-nicotinoylpiperidin-4-yl)-3-(4-(trifluoromethoxy)phenyl)urea as an off-white solid (940 mg, 76%). HPLC showed a purity of 98%. LCMS 409 [M+H], ¹H NMR (300 MHz, CD₃OD) δ 8.64-8.61 (m, 2H), 7.91-7.88 (m, 1H), 7.56-7.50 (m, 1H), 7.44-7.40 (m, 2H), 7.15-7.13 (m, 2H), 4.55-5.48 (m, 1H), 3.93-3.83 (m, 1H), 3.72-3.62 (m, 1H), 3.34-3.16 (m, 2H), 2.18-1.92 (m, 2H), 1.62-1.38 (m, 2H).

### Example 3

### Synthesis of 1-Adamantyl-3-(1-acetylpiperidin-4-yl)urea (3)

A reactor was charged with 1.00 mole-equivalent of 4-piperidinecarboxamide, 15.9 mole-equivalents of THF, and 1.23 mole-equivalents of N,N-(diisopropyl)ethylamine under a nitrogen atmosphere. The resulting mixture was cooled to 20° C. internal, and 1.10 mole-equivalents of acetic anhydride was added at such a rate as to maintain an internal temperature of less than 30° C. After addition was complete, the reaction mixture was stirred while maintaining an internal temperature of 20° C. The reaction contents was monitored until the amount of unreacted 4-piperidinecarboxamide was less than 1% relative to N-acetyl piperid-4-yl amide product (typically about 4-10 hours). The precipitated product was collected by filtration and washed with THF to remove excess (diisopropyl)ethylamine hydrochloride. The solid product was dried to constant weight in a vacuum oven under a nitrogen bleed while maintaining an internal temperature of 50° C. to afford the product as a white solid in 94% yield. ¹H NMR (CD₃OD): 4.48-4.58 (bd, 1H), 3.92-4.01 (bd, 1H), 3.08-3.22 (m, 1H), 2.62-2.74 (m, 1H), 2.44-2.53 (m, 1H), 2.12 (s, 3H), 1.88-1.93 (m, 2H), 1.45-1.72 (m, 2H); MS: 171 [M+H]⁺; m.p. 172-174° C.

A reactor was charged with 1.00 mole-equivalents of N-acetyl piperid-4-yl amide, 0.87 mole-equivalents of 1-adamantyl amine, and 49.7 mole-equivalents of acetonitrile, and the resulting mixture was heated to 75° C. internal under a nitrogen atmosphere. (Diacetoxyiodo)benzene (1.00 mole-equivalents) was charged portionwise in such a way that the reaction mixture was maintained between 75-80° C. internal. After the (diacetoxyiodo)benzene was added, the reaction mixture was heated to 80° C. internal. The reaction contents was monitored until the amount of unreacted 1-adamantyl amine was less than 5% relative to product N-(1-acetylpiperidin-4-yl)-N′-(adamant-1-yl)urea (typically about 1-6 hours). After completion, the reaction mixture was cooled to 25° C. internal, and approximately 24 mole-equivalents of solvent was distilled out under vacuum while maintaining internal temperature below 40° C. The reaction mixture was cooled with agitation to 0-5° C. internal and stirred for an additional 2 hours. The technical product was collected by filtration and washed with acetonitrile. The crude product was dried to constant weight in a vacuum oven under a nitrogen bleed maintaining an internal temperature of 50° C. The dried, crude product was slurried with water maintaining an internal temperature of 20° C. internal for 4 hours and then collected by filtration. The filter cake was washed with heptane under a nitrogen atmosphere then dried to constant weight in a vacuum oven under a nitrogen bleed maintaining an internal temperature of 70° C. to afford product as a white solid in 72% yield based on 1-adamantyl amine. ¹H NMR (DMSO-d₆): 5.65-5.70 (bd, 1H), 5.41 (s, 1H), 4.02-4.10 (m, 1H), 3.61-3.70, (m, 1H), 3.46-3.58 (m, 1H), 3.04-3.23 (m, 1H), 2.70-2.78 (m, 1H), 1.98 (s, 3H), 1.84 (s, 6H), 1.64-1.82 (m, 2H), 1.59 (s, 6H), 1.13-1.25 (m, 1H), 1.00-1.12 (m, 1H); MS: 320 [M+H]⁺; m.p. 202-204° C.

### Example 4

### Synthesis of ethyl 2-fluoro-8-(3-adamantylureido)octanoate (4)

6-Amino-1-hexanol (9.00 g, 7.67 mmol) was taken in 300 mL of THF/Water (1:1) and to it was added tBoc anhydride (18.0 g, 8.44 mmol) followed by sodium carbonate (19.0 g, 19.2 mmol). The reaction mixture was then stirred at room temperature for 3 hours. After completion of the reaction, the resulting mixture was poured into water and extracted with ethyl acetate (2×300 mL). The combined organic layers were washed with water and brine and dried over sodium sulfate. Evaporation of the organic layer gave 16 g (96%) of tent-butyl 6-hydroxyhexylcarbamate which was essentially pure and was used without further purification.

tent-Butyl 6-hydroxyhexylcarbamate (16 g) was dissolved in 500 mL of DCM and to it was added 24.0 g of PCC and 60 g of neutral alumina. The reaction mixture was stirred at room temperature, and the progress of the reaction was monitored by TLC. The reaction was complete after 6 hours. The reaction mixture was filtered, and the filtrate was washed with water several times. The organic layer was evaporated under reduced pressure, and the crude product was purified by flash chromatography using ethyl acetate:hexane (1:3) as eluent to give tert-butyl 6-oxohexylcarbamate (14.4 g, 91%) as colourless oil.

tert-Butyl 6-oxohexylcarbamate (5.00 g, 2.74 mmol) was dissolved in 70 mL of dry THF and cooled to −78° C., and to it was added 12 mL of n-BuLi (1.6 M in hexane) and the solution stirred for 1 hour at −78° C. Triethyl-2-fluoro-2-phosphonoacetate (6.60 g, 2.74 mmol) dissolved in 20 mL of dry THF was added slowly to the reaction mixture via a cannula and the reaction mixture was allowed to warm to room temperature. The reaction mixture was then stirred at room temperature for 6 hours, poured into saturated ammonium chloride solution (200 mL), and extracted with ethyl acetate (2×300 mL). After evaporation of the organic layer, the crude product was purified by flash chromatography using ethyl acetate:hexane (1:4) as eluent to afford (Z)-ethyl 8-(tert-butoxycarbonylamino)-2-fluorooct-2-enoate (6.0 g, 68%).

(Z)-Ethyl 8-(tert-butoxycarbonylamino)-2-fluorooct-2-enoate (6.00 g, 1.78 mmol) was taken in 50 mL of DCM and to it was added 15 mL of TFA. The resulting mixture was stirred at room temperature for 2 hours. The reaction mixture was poured into water and extracted with DCM. The organic layer was washed with water and sodium bicarbonate solution, and, after drying over sodium sulfate, solvent was evaporated under reduced pressure. The crude product was purified by flash chromatography using ethyl acetate:hexane (2:3) as eluent to give (Z)-ethyl 8-amino-2-fluorooct-2-enoate (4.0 g, 95%). ¹H NMR (DMSO-d₆): δ 5.90-6.00 (m, 1H); 5.00 (bs, 2H); 4.20 (q, 2H); 3.20 (t, 2H); 2.60 (m, 2H); 1.60-1.80 (m, 6H); 1.40 (t, 3H). Mass: 204 (M+1, 100%).

(Z)-Ethyl 8-amino-2-fluorooct-2-enoate of Example 1 (2.0 g, 1.0 mmol) was dissolved in 50 mL of DCM and to it was added adamantyl isocyanate (1.7 g, 1.0 mmol) followed by triethylamine (2 mL, 2 mmol). The reaction mixture was stirred at room temperature for 6 hours. After completion of the reaction, the DCM layer was phase separated and washed with water several times. Evaporation of solvent gave the crude product which was purified by flash chormatography using ethyl acetate:hexane (2:3) as eluent to give (Z)-ethyl 2-fluoro-8-(3-adamantylureido)oct-2-enoate (3.4 g, 88%) as white solid. ¹H NMR (CDCl₃): δ 5.90-6.00 (m, 1H); 4.20 (q, 2H); 4.00 (bs, 2H); 3.20 (t, 2H); 2.60 (m, 2H); 2.00-1.80 (m, 6H); 1.70-1.40 (15H); 1.40 (t, 3H). Mass: 381 (M+1,100%).

(Z)-ethyl 2-fluoro-8-(3-adamantylureido)oct-2-enoate (2.0 g, 0.66 mmol) was taken in 20 mL of methanol and to it was added 350 mg of Pd/C (10%), and the reaction mixture was stirred at room temperature for 1.5 hours under a hydrogen atmosphere. After the reaction was complete, it was filtered through celite, the celite layer was washed with methanol, and the combined organic layers evaporated under reduced pressure. The crude product was purified by flash chromatography using ethyl acetate:hexane (2:3) as eluent to give ethyl 2-fluoro-8-(3-adamantylureido)octanoate (1.7 g, 93%) as a white solid. ¹H NMR (CDCl₃): δ 5.10-5.00 (m, 1H); 4.20 (q, 2H); 4.00 (bs, 2H); 3.20 (t, 2H); 2.60 (m, 2H); 2.00-1.80 (m, 7H); 1.70-1.40 (m, 15H); 1.40 (t, 3H). Mass: 383 (M+1,100%).

### Example 5

### 2-fluoro-8-(3-adamantylureido)octanoic acid

2-Fluoro-8-(3-adamantylureido)octanoate of Example 4 is subjected to ester hydrolysis reaction well known in the art. For example, 2-Fluoro-8-(3-adamantylureido)octanoate is taken in 10 ml of methanol/THF/water mixture and to it is added 100 mg of LiOH. The reaction mixture is stirred at room temperature for about 2 hours. After completion of the reaction, the reaction mixture is filtered through celite, the celite layer is washed with methanol, and the combined organic layer is evaporated under reduced pressure. The crude product is purified by flash chromatography to afford 2-fluoro-8-(3-adamantylureido)octanoic acid.

### Example 6

### Treatment of Angiotensin II Infused Apolipoprotein E deficient Mice with Compound 2

Six-month old apolipoprotein E deficient mice were chronically infused with angiotensin II (1.44 mg/Kg/day) for 4 weeks to induce an abdominal aortic aneurysm (AAA) and accelerate atherosclerosis development. The mice were treated with Compound 2 (1.5 g/L in drinking water) or vehicle for 4 weeks. The results demonstrated that Compound 2 significantly reduced the rate of AAA formation and atherosclerotic lesion area. These effects were associated with a reduction of serum lipid, IL-6, murine IL-8 KC and IL-1α, and down-regulation of gene expressions of ICAM-1, VCAM-1 and IL-6 in the arterial wall. The present data demonstrate that treatment with an sEH inhibitor attenuates AAA formation and atherosclerosis development. The attendant down-regulation of inflammatory mediators and lipid lowering effects may both contribute to the observed vascular protective effects.

### Experimental Design and Surgical Procedures

Six-month old male apoE deficient mice (The Jackson Laboratory, Bar Harbor, Me.) fed a normal chow (Harlan Teklad diet #2018, Harlan Laboratories, Inc., Indianapolis, Ind.) were used in this study. Baseline blood pressure and body weight were measured before surgery. Animals were anesthetized by inhalation of 2% isoflurane. The left common carotid artery was carefully dissected via a midline neck incision under a dissecting microscope, and then ligated with a 6-0 silk ligature just proximal to its bifurcation. At the time of ligation, a minipump (model 2004, Durect Corp., Cupertino, Calif.) filled with Ang II (1.44 mg/Kg/day, Phoenix Pharmaceuticals, Burlingame, Calif.) was implanted subcutaneously. The animals were randomly divided into 2 groups; Vehicle: drinking water containing 5% hydroxypropyl-beta-cyclodextrin (HPBCD) or Compound 2 in drinking water containing 1.5 mg/mL Compound 2 in 5% HPBCD. Each experimental group included 11 animals. After 4 weeks of Ang II infusion, systolic blood pressure was measured in conscious mice using a tail-cuff system (Kent Scientific Corporation, Torrington, Conn.), and the animals were euthanized. Blood samples were collected via cardiac puncture for the measurement of a serum cholesterol profile (IDEXX Veterinary Services, West Sacramento, Calif.) and serum inflammatory panel (Murigenics, Hayward, Calif.) using a mouse cytokine/chemokine panel kit (Millipore, Billerica, Mass.), and tissues were removed for analysis (see below).

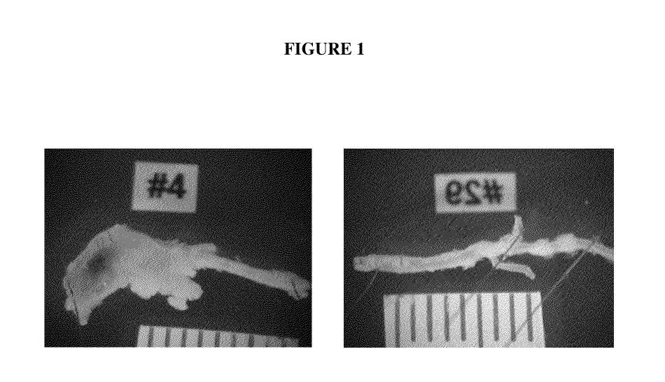

FIG. 1 illustrates that infusion of angiotensin II for 4 weeks induced abdominal aortic aneurysm (picture on left) in apolipoprotein E deficient mice, which can be partially prevented by the treatment with Compound 2 (picture on right).

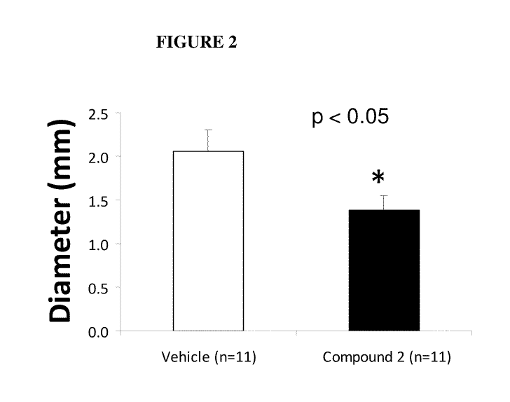

FIG. 2 illustrates an average diameter of the suprarenal aorta in angiotensin II infused apoE deficient mice treated with Compound 2 and with vehicle.

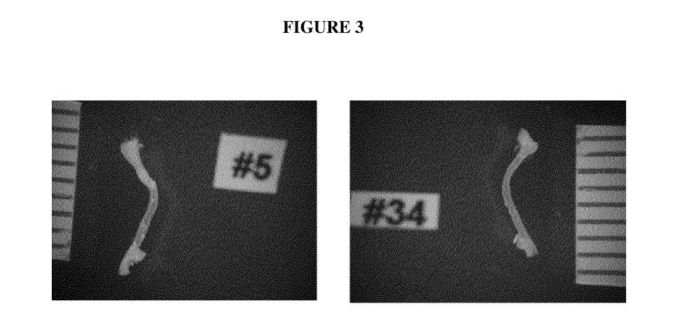

FIG. 3 illustrates that infusion of angiotensin II for 4 weeks exacerbated the atherosclerotic lesion development in the carotid artery (picture on left) in apolipoprotein E deficient mice. Treatment with Compound 2 significantly reduced the lesion area (picture on right).

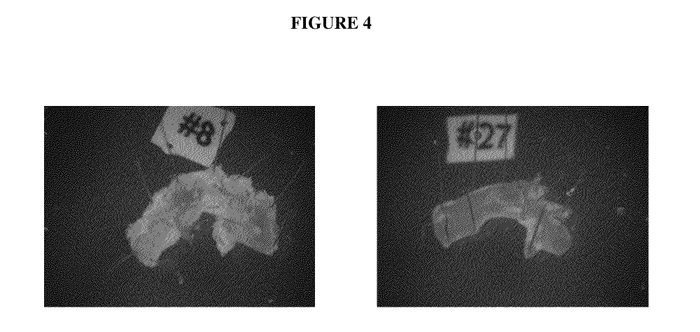

FIG. 4 illustrates that infusion of angiotensin II for 4 weeks exacerbated the atherosclerotic lesion development in the aortic arch (picture on left) in apolipoprotein E deficient mice. Treatment with Compound 2 significantly reduced the lesion area (picture on right).

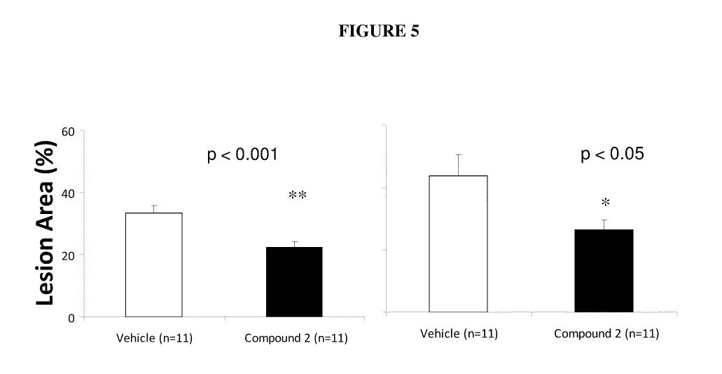

FIG. 5 illustrates an atherosclerotic lesion area in the right carotid aretery in angiotensin II infused apoE deficient mice treated with Compound 2 and with vehicle (graph on the left); and an atherosclerotic lesion area in the aortic arch in angiotensin II infused apoE deficient mice treated with Compound 2 and with vehicle (graph on the right).

### Compound 2 Attenuated Abdominal Aortic Aneurysm Formation

Chronic infusion of Ang II induced aneurysm formation in the abdominal aorta in 7 of 11 (64%) control apoE deficient mice. Treatment with Compound 2 reduced the incidence of aneurysm formation to 18% (2 of 11). The average outer diameter of the suprarenal aorta was significantly smaller in the Compound 2-treated mice than in the vehicle group. In mice that developed aneurysm, the severity measured by category score was also relatively less in the Compound 2-treated mice (majority with type 0 and I) compared to the vehicle group (majority with type III). There was no type VI aneurysm found in this study.

Histological staining showed that the aortas from the vehicle group had thick walls with intimal plaques, irregular media, and prominent adventitia. There were foci of acute hemorrhage present in the intima. The intima was occasionally disrupted by plaques of Mac-3-positive foam cells on the luminal side of the internal elastic lamina. Prussian blue staining showed iron accumulation co-localized with Mac-3-positive staining in the intima and adventitia. The thickness of the media was increased by extracellular matrix depositing between smooth muscle bundles and stained with trichrome as collagen. Elastin fibers in the media were discontinuous and irregularly oriented. The adventitia was markedly thickened by extracellular matrix that was predominately collagen. There was a modest increase in adventitial cellularity including fibroblasts and Mac-3-positive mononuclear cells. Segmental regions of the aortic wall showed replacement of the media and adventitia by thick bands of fibroblasts in a collagenous matrix. The aorta from Compound 2-treated animals had fewer intimal plaques and no evidence of macrophage and iron accumulation in the intima. These vessels did have medial changes including collagen deposition and some increase in elastin fibers, but the internal elastic lamina generally remained intact and retained a distinctive media of smooth muscle. The adventitia was relatively thin and composed of woven bands of collagen and had less macrophage and no iron accumulation.

### Compound 2 Reduced Atherosclerotic Lesions in the Aortic Arch and Non-Ligated Right Carotid Artery

The non-ligated right carotid artery displayed typical and severe fibrous-fatty lesions in the area proximal to the aortic arch and close to the bifurcation. Such atherosclerotic lesions were also observed in the aortic arch. Compound 2 treatment significantly reduced atherosclerotic lesion size in both the carotid artery and aortic arch.

### Compound 2 Had No Effect on Ligation-Induced Vascular Remodeling in the Carotid Artery

Ligation of the left carotid arteries for four weeks induced vascular remodeling, including neointima formation and adventitial proliferation, leading to expansive remodeling as measured by enlargement of vessel diameter. This was not affected by the Compound 2 treatment. The average diameter of the ligated carotid arteries was not significantly different between the two test groups.

### Compound 2 Down-Regulated the Expression of Pro-Inflammatory Mediators in the Aortic Tissue and in the Blood

Using the animal model as reported in Martin-McNulty B, et al., 17 Beta-estradiol attenuates development of angiotensin II-induced aortic abdominal aneurysm in apolipoprotein E-deficient mice. Arterioscler Thromb Vasc Biol. 2003; 23(9):1627-1632 and Tham D M, et al., Angiotensin II is associated with activation of NF-kappaB-mediated genes and downregulation of PPARs. Physiol Genomics. 2002; 11(1):21-30, treatment with Compound 2 significantly reduced the expression of pro-inflammatory genes such as VCAM-1, ICAM-1 and IL-6, but did not significantly affect the expression of IL-1α and PPARs measured in the ascending aortic tissue. Consistent with aortic gene expression, circulating protein levels of IL-6 and murine IL-8-KC were also significantly lower in Compound 2-treated mice than in the vehicle group. Interestingly, the serum IL-1α was reduced in Compound 2 group.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1. A method for treating inflammatory vascular disease in a subject, comprising administering to the subject an effective amount of a soluble epoxide hydrolase (sEH) inhibitor.

2. The method of claim 1, wherein said inflammatory vascular disease is selected from the group consisting of in-stent stenosis, coronary arterial disease, angina, acute myocardial infarction, acute coronary syndrome, chronic heart failure, peripheral arterial occlusive disease, critical limb ischemia, cardiac, kidney, liver or intestinal ischemia, renal failure, and cardiac hypertrophy.

3. The method of claim 1, wherein said inflammatory vascular disease is atherosclerosis.

4. The method of claim 1, wherein said inflammatory vascular disease is abdominal aortic aneurysm.

5. The method of claim 1, wherein said inflammatory vascular disease is vasculitis.

6. The method of claim 1, wherein said inflammatory vascular disease is carotid artery stenosis.

7. The method of claim 1, wherein said inflammatory vascular disease is a prelude to stroke.

8. The method of claim 1, wherein the sEH inhibitor is a compound of Formula (I) or a stereoisomer, tautomer, or pharmaceutically acceptable salt thereof:
R¹LC(=Q)NHR² (I)
wherein:
L is selected from the group consisting of a covalent bond, alkylene, O, S and NH;
Q is selected from the group consisting of O and S; and
R¹ and R² independently are selected from the group consisting of substituted alkyl, aryl, substituted aryl, heteroaryl, substituted heteroaryl, cycloalkyl, substituted cycloalkyl, heterocycloalkyl, and substituted heterocycloalkyl.

9. The method of claim 8, wherein R¹ is cycloalkyl, substituted cycloalkyl, phenyl or substituted phenyl.

10. The method of claim 8, wherein R² is substituted alkyl or substituted heterocycloalkyl.

11. The method of claim 8, wherein Q is O.

12. The method of claim 1, wherein the sEH inhibitor is a compound of Formula (II) or a stereoisomer, tautomer, or pharmaceutically acceptable salt thereof: 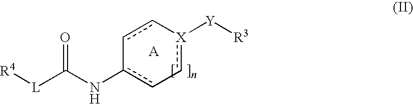
wherein:
L is selected from the group consisting of a covalent bond, alkylene, O, S and NH;
R³ is selected from the group consisting of alkyl, substituted alkyl, heteroaryl, substituted heteroaryl, heterocycloalkyl, and substituted heterocycloalkyl;
R⁴ is selected from the group consisting of aryl, substituted aryl, heteroaryl, substituted heteroaryl, cycloalkyl, substituted cycloalkyl, heterocycloalkyl, and substituted heterocycloalkyl;
n is 0, 1 or 2;
X is C, CH or N; provided that when X is C then n is 1 and ring A is phenyl; and
Y is selected from the group consisting of NH, O, C(═O)O, C(═O) and SO₂.

13. The method of claim 12, wherein R⁴ is adamantyl or substituted adamantyl.

14. The method of claim 12, wherein R⁴ is phenyl or substituted phenyl.

15. The method of claim 1, wherein the compound is of Formula (III), 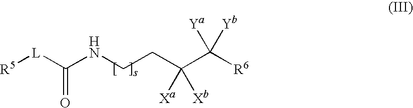
wherein
L is selected from the group consisting of a covalent bond, alkylene, O, S and NH;
R⁵ is selected from the group consisting of aryl, substituted aryl, heteroaryl, substituted heteroaryl, cycloalkyl, substituted cycloalkyl, heterocycloalkyl, and substituted heterocycloalkyl;
s is 0-10;
R⁶ is selected from the group consisting of —OR⁷, —CH₂OR⁷, —COR⁷, —COOR⁷, —CONR⁷R⁸, or a carboxylic acid isostere;
R⁷ and R⁸ independently are selected from the group consisting of hydrogen, alkyl, substituted alkyl, cycloalkyl, substituted cycloalkyl, heterocycloalkyl, substituted heterocycloalkyl, aryl, substituted aryl, heteroaryl, and substituted heteroaryl; or R⁷ and R⁸ together with the nitrogen atom bound thereto form a heterocycloalkyl ring having 3 to 9 ring atoms, and wherein said ring is optionally substituted with alkyl, substituted alkyl, heterocyclic, oxo or carboxy; and
each of X^{a}, X^{b}, Y^{a}, and Y^{b} is independently selected from the group consisting of hydrogen, C₁-C₄ alkyl, substituted C₁-C₄ alkyl, and halo.

16. The method of claim 15, wherein R⁵ is adamantyl or substituted adamantyl.

17. The method of claim 15, wherein R⁵ is phenyl or substituted phenyl.

18. The method of claim 15, wherein at least one of Y^{a} and Y^{b} is halo.

19. The method of claim 1, wherein the compound is of Formula (IV): 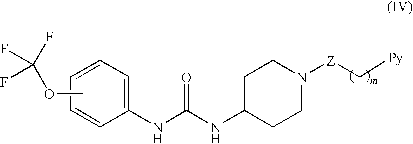
wherein
Z is CO or SO₂;
m is 0-2; and
Py is pyridyl or substituted pyridyl provided that when m is 0 then Z is on the 3- or 4-position of the pyridyl ring.

20. The method of claim 19, wherein Z is CO.

21. The method of claim 19, wherein m is 0.

22. The method of claim 19, wherein m is 1.

23. The method of claim 19, wherein m is 0 and Z is on the 3-position of the pyridyl ring.

24. The method of claim 1, wherein the compound is selected from the group consisting of 1-adamantyl-3-(1-(methylsulfonyl)piperidin-4-yl)urea, 1-(1-nicotinoylpiperidin-4-yl)-3-(4-(trifluoromethoxy)phenyl)urea, 1-adamantyl-3-(1-acetylpiperidin-4-yl)urea, ethyl 2-fluoro-8-(3-adamantylureido)octanoate and 2-fluoro-8-(3-adamantylureido)octanoic acid.

25. The method of claim 1, wherein the compound is selected from the group consisting of

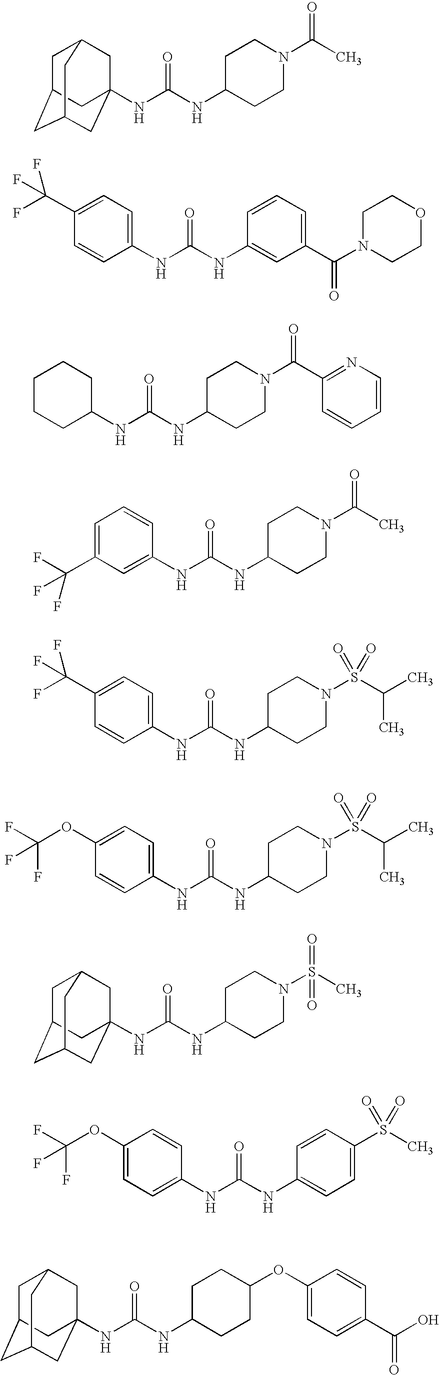

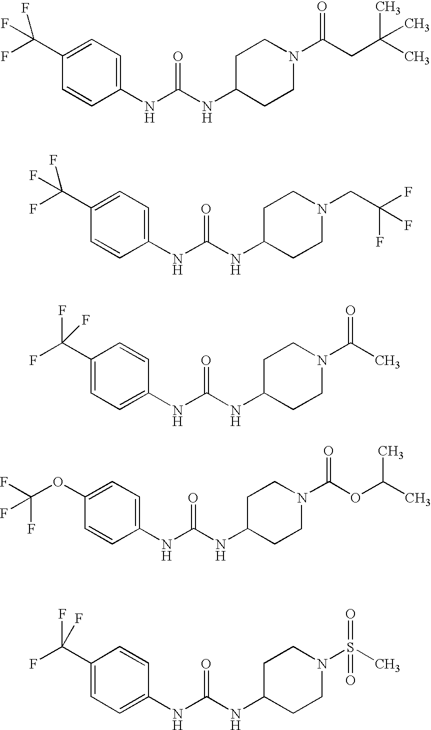

26. The method of claim 1, wherein the compound is 1-(1-nicotinoylpiperidin-4-yl)-3-(4-(trifluoromethoxy)phenyl)urea.

27. The method of claim 1, wherein the compound is 1-adamantyl-3-(1-acetylpiperidin-4-yl)urea.

28. The method of claim 1, wherein the compound is ethyl 2-fluoro-8-(3-adamantylureido)octanoate.

29. The method of claim 1, wherein the compound is 2-fluoro-8-(3-adamantylureido)octanoic acid.

30. A method of treating a disease mediated at least in part by angiotensin II in a subject, comprising administering to the subject an effective amount of a sEH inhibitor.

31. A method of identifying a disease treatable by a sEH inhibitor in a diseased subject, wherein said method comprises:
a) assaying a level of angiotensin II in said diseased subject to determine if said level is abnormal; and
b) treating said diseased subject identified in a) above with abnormal level of angiotensin II with an sEH inhibitor.

32. A stent comprising a surface, wherein the surface comprises a biodegradable composition coating comprising an sHE inhibitor.

33. The stent of claim 32, wherein the biodegradable composition is a polymer.
